(19) United States
(12) Patent Application Publication
(10) Pub. No.: US 2007/0123460 A1
(43) Pub. Date: May 31, 2007

(54) Probiotic compounds from Lactobacillus GG and uses therefor

(75) Inventors: Eugene B. Chang, Chicago, IL (US); Elaine O. Petrof, Chicago, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE UNIVERSITY OF CHICAGO, Chicago, IL (US)

(21) Appl. No.: 11/581,719

(22) Filed: Oct. 16, 2006

Related U.S. Application Data
(63) Continuation-in-part of application No. PCT/US05/13646, filed on Apr. 20, 2005.

(51) Int. Cl. A61K 38/16; C07K 14/195

(52) U.S. Cl. 514/12; 530/350

(57) ABSTRACT

The invention provides methods and compositions for the treatment of inflammatory disorders, such as inflammatory bowel diseases (IBDs). The use of bacteria-free, probiotic-derived compounds instead of live bacteria provides a safety advantage over the use of live bacteria. In addition, the administration of isolated compounds will provide more reliable dosing, greater simplicity, and improved consistency than is found in administering probiotics, which is dependent on both establishing and maintaining bacterial colonization.

The government owns rights in the invention pursuant to grant numbers DK47722, DK42086, and K08 DK064840-01 from the National Institutes of Health.

### FIELD OF THE INVENTION

The invention relates generally to the field of inflammatory disorders. More particularly, it concerns inflammatory bowel disorders or diseases, such as ulcerative colitis and Crohn's disease. An implementation of the invention relates to the identification and characterization of novel bioactive compounds derived from Lactobacillus GG (LGG) and the use of these compounds to treat inflammatory bowel disease.

### BACKGROUND

Inflammatory bowel diseases (IBDs) are a group of chronic disorders that affect the digestive tract of susceptible individuals. The extent and severity of mucosal injury in IBD is determined by the disequilibrium between inflammation-induced injury-versus reparative and cytoprotective processes. Exemplary IBDs, such as ulcerative colitis and Crohn's disease, cause inflammation or ulceration of the digestive tract. The unfortunate combination of genetic background, exposure to environmental factors, or colonization by certain inciting commensal bacteria, can result in the development of IBD in susceptible individuals.

Changing the gut flora of IBD patients with probiotic agents has received some attention as a therapeutic strategy. In recent in vitro and in vivo studies, various probiotic formulations have been shown to be effective in either preventing or mitigating intestinal mucosal inflammation associated with experimental colitis (Madsen et al., 2001; Gionchetti et al., 2000b; Campierei et al., 2000; complete citations are provided in the list of references). Furthermore, probiotics appear to reduce the rate of malignant transformation of colonic mucosa in the setting of chronic inflammation (Wollowski et al., 2001). A number of preliminary clinical trials have shown that probiotics are effective in the treatment of pouchitis and IBD. Several multicenter clinical trials are also under way to determine the effectiveness of these agents and to optimize dosages in IBD patients. Despite these promising results, the mechanism(s) of probiotic action remains unclear (Shanahan, 2002), and the use of live probiotic organisms presents risks of infection and other untoward consequences.

Ulcerative colitis, an exemplary IBD, causes inflammation and ulceration of the inner lining of the colon and rectum. It rarely affects the small intestine except for the end that connects to the colon, called the terminal ileum. Ulcerative colitis may also be called colitis or proctitis. Ulcerative colitis may occur in people of any age, but most often it starts between ages 15 and 30. Ulcerative colitis affects men and women equally and appears to run in some families. Theories about what causes ulcerative colitis abound, but none have been proven. A popular theory is that the body's immune system reacts to a virus or a bacterium by causing ongoing inflammation in the intestinal wall.

The most common symptoms of ulcerative colitis are abdominal pain and bloody diarrhea. Patients also may experience fatigue, weight loss, loss of appetite, rectal bleeding, and loss of body fluids and nutrients. About half of patients have mild symptoms. Others suffer frequent fever, bloody diarrhea, nausea, and severe abdominal cramps. Ulcerative colitis may also cause problems such as arthritis, inflammation of the eye, liver disease (hepatitis, cirrhosis, and primary sclerosing cholangitis), osteoporosis, skin rashes, and anemia. No one knows for sure why problems occur outside the colon. Scientists think these complications may occur when the immune system triggers inflammation in other parts of the body. Some of these problems go away when the colitis is treated.

Treatment for ulcerative colitis depends on the seriousness of the disease. Most people are treated with medication. In severe cases, a patient may need surgery to remove the diseased colon. Some people whose symptoms are triggered by certain foods are able to control the symptoms by avoiding foods that upset their intestines, like highly seasoned foods, raw fruits and vegetables, or milk sugar (lactose). Some people have remissions that last for months or even years. However, most patients' symptoms eventually return.

About 25-40% of ulcerative colitis patients must eventually have their colons removed because of massive bleeding, severe illness, rupture of the colon, or risk of cancer. Sometimes the doctor will recommend removing the colon if medical treatment fails or if the side effects of corticosteroids or other drugs threaten the patient's health.

Crohn's disease differs from ulcerative colitis in that it may affect any part of the digestive tract. It causes inflammation and ulcers that may affect the deepest layers of lining of the digestive tract. Anti-inflammatory drugs, such as 5-aminosalicylates (e.g., mesalamine) or corticosteroids, are typically prescribed, but are not always effective. Immunosuppression with cyclosporine is sometimes beneficial for patients resistant to or intolerant of corticosteroids.

Nevertheless, surgical correction is eventually required in 90% of patients with Crohn's disease; 50% undergo colonic resection. (Leiper et al., 1998; Makowiec et al., 1998). The recurrence rate after surgery is high, with 50% requiring further surgery within 5 years. (Leiper et al., 1998; Besnard et al., 1998).

Current concepts regarding the etiopathogenesis of IBD suggest that there is a disequilibrium between the processes of cytoprotection and wound healing and the pro-inflammatory pathways, the net result of which culminates in a state of proinflammatory overactivity and resultant damage to the intestinal mucosa (Chang, 1999; Podolsky, 2002). Central to preserving mucosal integrity is maintenance of epithelial barrier function, as evidenced by the fact that altered tight junction structure resulting in impaired barrier function is thought to contribute to the clinical sequelae of ulcerative colitis (Schmitz et al., 1999).

Therapies can be used to induce and maintain remission, and to improve the quality of life for people with an inflammatory disease or disorder, such as ulcerative colitis. Several types of drugs are currently available.

Aminosalicylate drugs, such as those that contain 5-aminosalicylic acid (5-ASA), help control inflammation. Sulfasalazine is a combination of sulfapyridine and 5-ASA and is used to induce and maintain remission. The sulfapyridine component carries the anti-inflammatory 5-ASA to the intestine. However, sulfapyridine may lead to side effects such as nausea, vomiting, heartburn, diarrhea, and headache. Other 5-ASA agents such as olsalazine, mesalamine, and balsalazide, have a different carrier, offer fewer side effects, and may be used by people who cannot take sulfasalazine. 5-ASAs are given orally, through an enema, or in a suppository, depending on the location of the inflammation in the colon. Most people with mild or moderate ulcerative colitis are treated with this group of drugs first.

Corticosteroids, such as prednisone and hydrocortisone, also reduce inflammation. They may be used by people who have moderate to severe ulcerative colitis or who do not respond to 5-ASA drugs. Corticosteroids can be given orally, intravenously, through an enema, or in a suppository. These drugs can cause side effects such as weight gain, acne, facial hair, hypertension, mood swings, and an increased risk of infection. For this reason, they are not recommended for long-term use.

Immunomodulators, such as azathioprine and 6-mercapto-purine (6-MP), reduce inflammation by affecting the immune system. They are used for patients who have not responded to 5-ASAs or corticosteroids or who are dependent on corticosteroids. However, immunomodulators are slow-acting and it may take up to 6 months before the full benefit is seen. Patients taking these drugs are monitored for complications including pancreatitis and hepatitis, a reduced white blood cell count, and an increased risk of infection. Cyclosporine A may be used with 6-MP or azathioprine to treat active, severe ulcerative colitis in people who do not respond to intravenous corticosteroids. In addition to the above, other drugs may be given to relax the patient or to relieve pain, diarrhea, or infection.

Lactobacillus GG has been used successfully in the treatment of acute and rotavirus diarrhea in infants and children (Isolauri et al., 1991; Majamaa et al., 1995), and also in the treatment of antibiotic-associated diarrhea resulting from alterations in the normal commensal flora (Arvola et al., 1999; Kalliomaki et al., 2003; Szajewska et al., 2001; Vanderhoof et al., 1999). Finally, Lactobacillus GG has been shown to decrease the level of tumor burden in a murine model of colon cancer, suggesting that this probiotic strain may also possess anti-carcinogenic activity (Goldin et al., 1996).

The induction of cellular heat shock protein (Hsp) expression, as occurs after thermal stress such as fever, is a well-described mechanism by which cells are able to defend themselves against further injury. This phenomenon, known as “stress tolerance” is highly conserved throughout evolution and across all species (Parsell et al., 1993). Inducible heat shock proteins confer protection on cells in the face of a variety of different types of stress, ranging from thermal and osmotic stress to oxidative and inflammatory stressors (Parsell et al., 1993). Overexpression of Hsp72 in intestinal epithelial cells has been shown to increase viability and protection against oxidative injury from monochloramine (Musch et al., 1996; Musch et al., 1999), a pathophysiologically relevant reactive oxygen metabolite produced in large quantities during inflammation when hypochlorous acid released by innate and inflammatory cells reacts with ammonia (Grisham et al., 1990). In intestinal epithelial cells, the inducible heat shock proteins Hsp72 and Hsp25 reportedly fortify the epithelial barrier against damage from a variety of injurious insults, thus preserving tight junction and barrier function (Liu et al., 2003; Musch et al., 1996; Musch et al., 1999; Ropeleski et al., 2003; Urayama et al., 1998). Hsp25 has also been reported to stabilize the actin cytoskeleton.

Through the use of sense and antisense transfection experiments, it has been shown that heat shock proteins play a central role in providing cytoprotection to epithelial cells, as illustrated by their ability to protect epithelial barrier function under conditions of oxidative stress (Ropeleski et al., 2003; Urayama et al., 1998). Inducible heat shock proteins (Hsp) belong to a family of highly conserved proteins that play an important role in protecting cells against physiologic and pathogenic stressors in the environment. Under conditions of stress such as heat, exposure to heavy metals, and toxins, ischemia/reperfusion injury, or oxidative stress from inflammation, Hsp induction is both rapid and robust. Induction of heat shock proteins by a mild “stress” confers protection against subsequent insult or injury, which would otherwise lead to cell death. This well-described phenomenon is known as “stress tolerance” (Parsell et al., 1993).

In intestinal epithelial cells, inducible heat shock proteins convey a degree of cytoprotection against stressors such as inflammatory cell-derived oxidants and thermal stresses (e.g., fever); inducible Hsps also preserve the integrity of intestinal epithelial cell barrier function under hostile conditions (Chang, 1999; Musch et al., 1996; Musch et al., 1999). The induction of heat shock proteins in intestinal epithelial cells prolongs viability under conditions of stress (Musch et al., 1996) and preserves tight junctions as measured by transepithelial resistance (Musch et al., 1999).

Live LGG bacteria have also been reported to inhibit the p38 MAP kinase, although no effect is seen on any of the MAP kinases with conditioned media alone (Yan et al., 2002). The conditioned media employed was prepared by growing the bacteria in MRS broth and then pelleting, rinsing, and resuspending the bacteria in tissue culture media to allow for an additional two hours of growth, followed by filtering before use.

There is growing interest in the use of probiotics, which are defined as ingestible microorganisms having health benefit beyond their intrinsic nutritive value, in the treatment of a variety of gastrointestinal ailments including inflammatory bowel diseases (Gionchetti et al., 2000a), irritable bowel syndrome (Niedzielin et al., 2001), pouchitis (Gionchetti et al., 2000b; Gionchetti et al., 2003), as well as rotavirus and antibiotic-associated diarrhea (Isolauri et al., 1991; Majamaa et al., 1995; Arvola et al., 1999). Although little is known about their mechanisms of action, probiotics appear to have protective, trophic, and anti-inflammatory effects on bowel mucosa.

The probiotic organism Lactobacillus GG has been used successfully in the treatment of acute and rotavirus diarrhea in infants and children (Isolauri et al., 1991; Majamaa et al., 1995), and also in the treatment of antibiotic-associated diarrhea (Arvola et al., 1999; Kalliomaki et al., 2003). Rotavirus infection requires an initial interaction of the VP4 spike protein with the surface of the epithelial cell and the C-terminal fragment, VP5*, is thought to be responsible for membrane permeabilization of the cell, which is necessary for viral entry (Zarate et al., 2000).

Probiotics may also prove useful in the treatment and prevention of atopic disease (Kalliomaki et al., 2003). In several animal models, the use of probiotics appears protective against C. parvum (Alak et al., 1997), H. pylori (Hamilton-Miller, 2003), and Candida (Wagner et al., 1997) infections. In addition, Lactobacillus GG has been shown to decrease the level of tumor burden in a murine model of colon cancer, suggesting that this probiotic strain may also possess anti-carcinogenic activity (Goldin et al., 1996).

Although probiotics appear to improve the course of many illnesses, probiotic mechanisms of action are poorly understood, and this is a recognized weakness in the field. Only in recent years has an attempt been made to understand the mechanisms behind their actions and interactions with the host cell. Many different possible mechanisms have been proposed, including upregulation of mucus production, improvement in epithelial barrier function, increase in IgA production, and increased competition for adhesion sites on intestinal epithelia, as well as the production of organic acids, ammonia, hydrogen peroxide, and bacteriocins which inhibit the growth of pathogenic bacteria (Cleveland et al., 2001; Lee et al., 2000; Lee et al., 2003; Madsen et al., 2001; Malin et al., 1996).

Changing the gut flora of IBD patients with probiotic agents is being studied as a therapeutic strategy, but the mechanisms of probiotic action remain unclear. Moreover, the clinical efficacy of probiotics is highly dependent on the ability to establish and maintain bacterial colonization, is dependent on the bacteria for reliable consistent production of the active agents, and is limited by the unregulated composition of formulations and homeopathic delivery of those agents. Moreover, the use of live bacteria presents unavoidable risks of infection and disease. Thus, there is a need for isolated, bioactive probiotic factors and for more effective therapies to prevent or treat inflammatory disorders such as inflammatory bowel disease.

### SUMMARY OF THE INVENTION

The invention satisfies at least one of the aforementioned needs in the art by providing bioactive cytoprotective compounds that are secreted by Lactobacillus GG and that induce the expression of heat shock proteins. The cytoprotective effects of the heat shock proteins can bolster a cell's defenses against inflammation. Thus, the compounds of the invention provide methods and compositions for the treatment of IBD and other inflammatory disorders.

Without wishing to be bound by theory, it is noted that the protective and beneficial effects of probiotics on intestinal epithelial cell function indicate that one of the mechanisms of probiotic action may involve the induction of cytoprotective heat shock proteins. This disclosure reveals that peptides synthesized by the probiotic LGG possess the ability to induce cytoprotective heat shock proteins in murine intestinal epithelial cells in a time- and concentration-dependent manner involving transcriptional regulation by the transcription factor HSF-1. Of further interest, the findings indicate that the conditioned media from LGG not only provides protection against oxidant stress and upregulates epithelial cell heat shock proteins, they also modulate signal transduction pathways.

In one aspect, the invention provides a composition comprising an isolated cytoprotective compound derived from Lactobacillus GG, such as a Lactobacillus GG-conditioned medium. As used herein, “derived from” means ultimately obtained therefrom, either by direct isolation or, based on characteristics such as those disclosed herein or as determined using routine procedures in view of the disclosure herein. The compound is obtained using any technique known in the art, such as recombinant expression, chemical synthesis, and the like. In certain embodiments, the cytoprotective compound induces the expression of at least one heat shock protein. In preferred embodiments, the cytoprotective compound induces the expression of at least one of Hsp25 and Hsp72. In some embodiments of the invention, the cytoprotective compound is a protein. There are also embodiments in which the protein is heat stable. As used herein, “heat stable” refers to a protein capable of retaining detectable activity after boiling in water for 20 minutes. The activity of a cytoprotective protein of the invention can be determined by assaying its ability to induce the expression of at least one heat shock protein, such as Hsp25 or Hsp72. In some embodiments, the protein is acid stable. As used herein, an “acid stable” protein refers to a protein that is most active at a pH below 7.0, where activity is determined by the protein's ability to induce expression of Hsp25. In some embodiments, the protein has a molecular weight of less than 10 kDa. In a preferred embodiment, the cytoprotective compound is a protein that is heat stable, acid stable, and has a molecular weight of less than 10 kDa. In some embodiments, the cytoprotective compound protects an epithelial cell from a stress selected from the group consisting of heat and oxidation. In another preferred embodiment, the isolated cytoprotective compound is a protein having a capability of being isolated from Lactobacillus GG, a capability of inducing the expression of Hsp25 and Hsp72 in an epithelial cell such as an intestinal epithelial cell, a molecular weight of less than 10 kDa, and being both acid stable and heat stable.

Another aspect of the invention provides methods for treating a subject, such as a human patient, with an inflammatory disorder comprising administering to the patient a therapeutically effective amount or dose of an isolated cytoprotective compound derived from a Lactobacillus GG-conditioned medium. As used herein, a “therapeutically effective amount” is an amount that has a detectably beneficial effect in preventing, ameliorating or affecting the course of an inflammatory disorder, such as by inhibiting, or retarding the rate of, the development of such a disorder. An exemplary inflammatory disorder suitable for treatment according to this method is an inflammatory bowel disease, e.g, Crohn's disease and ulcerative colitis. In some embodiments, the cytoprotective compound induces the expression of at least one heat shock protein, such as Hsp25 and/or Hsp72.

A related aspect of the invention is drawn to a method for ameliorating a symptom of, or associated with, an inflammatory disorder comprising administering a therapeutically effective dose of an isolated cytoprotective compound derived from Lactobacillus GG, such as a Lactobacillus GG medium, to a subject, such as a human patient. An exemplary embodiment of this aspect of the invention is a method for ameliorating a symptom of an inflammatory bowel disease, such as Crohn's disease or ulcerative colitis. Related thereto is an aspect of the invention providing a method of preventing an inflammatory disorder comprising administering to the subject, such as a human patient, an isolated cytoprotective compound derived from Lactobacillus GG, such as an isolated cytoprotective compound present in a Lactobacillus GG-conditioned medium.

The inflammatory disorder may be an autoimmune disorder. Examples of autoimmune disorders that may be treated according to the invention include rheumatoid arthritis, juvenile rheumatoid arthritis, osteoarthritis, psoriatic arthritis, atopic dermatitis, eczematous dermatitis, psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, leprosy reversal reactions, erythema nodosum leprosum, autoimmune uveitis, polychondritis, Stevens-Johnson syndrome, lichen planus, sarcoidosis, primary biliary cirrhosis, uveitis posterior, interstitial cystitis, or interstitial lung fibrosis.

In a preferred embodiment, the inflammatory disorder is an inflammatory bowel disease. In an exemplary embodiment, the inflammatory bowel disease is Crohn's disease. In another exemplary embodiment of the invention the inflammatory bowel disease is ulcerative colitis. In still other embodiments, the compound being administered induces the expression of at least one heat shock protein, such as Hsp25 and/or Hsp72.

In methods of treating, ameliorating a symptom associated with, or preventing an inflammatory disorder, the invention comprehends administering an effective amount or dose of a pharmaceutical composition comprising a cytoprotective compound as disclosed herein to a subject such as a human patient. Such pharmaceutical compositions are described below.

Another aspect of the invention provides methods for inducing the expression of at least one heat shock protein, such as Hsp25 and/or Hsp72, in a cell comprising contacting the cell with an isolated cytoprotective compound derived from Lactobacillus GG, such as a cytoprotective compound present in a Lactobacillus GG-conditioned medium. In certain embodiments, the invention provides methods of inducing the expression of one or both of Hsp25 and Hsp72 in a cell comprising contacting the cell with a cytoprotective compound isolated from Lactobacillus GG or a Lactobacillus GG-conditioned medium. In certain embodiments, the isolated cytoprotective compound contacts an epithelial cell, such as an intestinal epithelial cell. In other embodiments, the cell is an immune cell, such as a dendritic cell.

Another aspect of the invention provides a pharmaceutical composition comprising an isolated cytoprotective compound derived from Lactobacillus GG, such as would be found in, or purified from, a Lactobacillus GG-conditioned medium, and at least one pharmaceutically acceptable excipient. In certain embodiments, the pharmaceutically acceptable excipient is polyethylene glycol. The cytoprotective compound, or bioactive agent, is in “isolated” form, which means that it has been separated from as least one protein with which it is naturally found in Lactobacillus GG cells or culture medium. In some embodiments according to this aspect of the invention, the compound induces the expression of at least one heat shock protein, such as Hsp25 and/or Hsp72.

In another aspect, the invention provides a method of producing an isolated cytoprotective compound comprising the steps of obtaining Lactobacillus GG and isolating a cytoprotective compound from Lactobacillus GG. As noted above, an isolated form of the cytoprotective compound means that the compound has been separated from as least one protein with which it is naturally found in Lactobacillus GG cells or culture medium. In obtaining the Lactobacillus GG, one may acquire a mass of such cells, or one may grow or culture such cells in a suitable medium, such as MRS, thereby obtaining the cells in a conditioned medium. Contemplated in one embodiment is a culturing period of at least 8 hours to ensure production of the cytoprotective compound. In certain embodiments, the cytoprotective compound is a protein. In some embodiments, the cytoprotective compound is heat stable and/or acid stable and/or has a molecular weight less than 10 kilodaltons (kDa). In yet another aspect, the method further comprises characterizing and/or identifying the cytoprotective compound.

Those skilled in the art will be familiar with methods for isolating proteins. For example, the cytoprotective proteins of the invention may be isolated by HPLC, FPLC, hydrophobic LC, ion exchange LC, ligand/affinity LC, size exclusion LC, thin layer chromatography, membrane filtration, isoelectric focusing, or polyacrylamide gel electrophoresis, or any combination of these methods.

Methods for characterizing the cytoprotective properties of the compounds of the invention will be well known to those of skill in the art. Indicators of cytoprotective activity include, for example, the ability to induce the expression of heat shock proteins, and the ability to reduce cell damage and/or promote wound healing in a subject with an inflammatory disorder. Thus, one approach to characterizing the compounds of the invention is to assay the induction of heat shock proteins. Another approach to characterizing the compounds of the invention would be to assay a compound's ability to reduce cell damage and/or promote wound healing in a subject.

Methods for identifying proteins are known to those skilled in the art. For example, the cytoprotective proteins may be identified by subjecting the samples to 6N hydrolysis followed by HPLC-Mass sequencing to identify all the individual amino acids that constitute the peptide of interest. Additionally, internal amino acid sequences can be determined from readily identified tryptic fragments. The amino acid sequence of these fragments may be determined by matrix-assisted laser desorption ionization—time of flight mass spectrometry (MALDI-TOF). The amino acid sequences are then compared with the relevant databases (e.g., SwissProt, Genbank) to identify the protein or peptide of interest.

In another aspect, the methods of the invention further comprise obtaining more cytoprotective compound. More cytoprotective compound may be obtained by any method known to those skilled in the art. For example, more cytoprotective compound may be obtained by isolation from Lactobacillus GG or Lactobacillus GG-conditioned media. Alternatively, more cytoprotective compound may be obtained by expression of a recombinant DNA encoding the cytoprotective compound.

In another aspect, the methods of the invention further comprise placing the more cytoprotective compound in a pharmaceutical composition. In certain aspects, the methods further comprise administering the pharmaceutical composition to a subject with an inflammatory disorder. The subject may be a mammal. Preferably the subject is a human.

In another aspect according to the invention, an isolated polynucleotide is provided that encodes a polypeptide cytoprotective compound, wherein the encoded polypeptide is characterized by the following properties: a capability of being isolated from Lactobacillus GG; a capability of inducing expression of Hsp25 and Hsp72 in intestinal epithelial cells; a molecular weight of less than 10 kDa; and the properties of being acid stable and heat stable.

A further aspect of the invention is drawn to the composition comprising the isolated cytoprotective compound as described above, wherein the compound activates a signal transduction pathway in an epithelial cell resulting in expression of a heat shock protein selected from the group consisting of Hsp25 and Hsp72. Unless otherwise indicated, Hsp70 is a synonym of Hsp72, as would be known in the art, although Hsp72 contains a more precise estimate of the mass of the protein. In some embodiments, the activation is mediated by Heat Shock Factor-1 (HSF-1). In some embodiments, the signal transduction pathway comprises a kinase selected from the group consisting of MAP kinase, SAP kinase, ERK1 and ERK2. In certain embodiments, the pathway activation comprises activation of a kinase selected from MAP kinase, SAP kinase, ERK1 and ERK2.

In a related aspect, the invention provides a method of activating a signal transduction pathway in an epithelial cell comprising contacting the cell with an effective amount of an isolated cytoprotective compound as disclosed herein. In some embodiments, the signal transduction pathway comprises a kinase selected from the group consisting of MAP kinase, SAP kinase, ERK1 and ERK2. In some embodiments, the isolated cytoprotective compound is administered in the form of a pharmaceutical composition.

Yet another aspect of the invention is drawn to a method of preventing oxidant injury comprising administering an effective amount of an isolated cytoprotective compound as disclosed herein, or a pharmaceutical composition comprising such a compound, to a cell, such as an epithelial cell.

Still another aspect according to the invention is a method of stabilizing a cytoskeleton comprising administering an effective amount of the isolated cytoprotective compound as disclosed herein, or a pharmaceutical composition comprising such a compound, to a cell, such as an epithelial cell.

Another aspect of the invention is a method of preventing an inflammatory disorder comprising administering an effective dose of the pharmaceutical composition disclosed herein to a subject. A preferred subject is a human patient.

Still another aspect according to the invention is a kit comprising the pharmaceutical composition disclosed herein and a protocol for administration of the composition to a subject. Any administration protocol known in the art as suitable for the intended purpose is contemplated by the invention. A preferred subject is a human patient.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the standard deviation of error for the device or method being employed to determine the value.

The words “a” and “an,” when used in conjunction with the word “comprising” in the claims or specification, denotes one or more, unless specifically noted.

Other features and advantages of the invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWING

The following drawing forms part of the present specification and is included to further demonstrate certain aspects of the invention. The invention may be better understood by reference to one or more of the figures of the drawing in combination with the detailed description of specific embodiments presented herein.

FIG. 1: LGG-conditioned media induces Hsp25 and Hsp72 in colonic epithelial cells in a time- and concentration-dependent manner. FIG. 1A shows the LGG-CM induction time course (600 μl LGG-CM/well). FIG. 1B depicts the LGG-CM dose-response relationship with treatment for 16 hours.

FIG. 2: Induction of heat shock proteins by LGG-conditioned media involves HSF-1.

FIG. 3: Bioactive factors in LGG-conditioned media appear to be of small molecular weight.

FIG. 4: Bioactive factors in LGG-conditioned media appear to be heat stabile and most active at acidic pH.

FIG. 5: Bioactive factors in LGG-conditioned media are inactivated by pepsin.

FIG. 6: Bioactive factors in LGG-conditioned media are inactivated by DTT.

FIG. 7: Histogram of time course of LGG-CM induced expression of heat shock proteins as revealed by real-time PCR. FIG. 7A: induction of Hsp72; FIG. 7B: induction of Hsp25.

FIG. 8: Electrophoretic mobility shift assays, using anti-HSF-1 and anti-HSF-2 antibodies as probes/binding agents, demonstrating that the induction of heat shock proteins by LGG-CM is at least partly transcriptional in nature.

FIG. 9: Scatter plots demonstrating that heat shock proteins are the most dramatically upregulated epithelial cell genes after exposure to LGG-CM.

FIG. 10: Electrophoretogram of washout experimental results demonstrating that the heat shock protein induction signal is rapidly transmitted upon exposure of LGG-CM to epithelial cells (FIG. 10A) and is mediated by at least one signal transduction pathway (FIG. 10B).

FIG. 11: Histogram demonstrating the protective effect of LGG-CM on epithelial cells challenged by an oxidant stress as revealed by viability testing with ⁵¹Cr (FIG. 11A) or by G/F actin assay of cytoskeletal integrity (FIG. 11B).

FIG. 12: Demonstration of the physical properties of a cytoprotective factor(s) derived from Lactobacillus GG. Electrophoretogram showing susceptibility of the factor(s) to pepsin (FIG. 12A), electrophoretic sizing of the factor(s) (FIG. 12B), and electrophoretic evidence of heat stability of factor(s).

FIG. 13: Electrophoretogram showing stability of cytoprotective factor(s) of LGG-CM as a function of pH (FIG. 13A) and partial renaturation of factor(s) upon re-acidification (FIG. 13B).

FIG. 14: LGG-CM induces YAMC cell Hsp25 and Hsp72 in a time and concentration-dependent fashion. YAMC cells were untreated (NoTx), treated with MRS broth (1:10 dilution), or varying dilutions of LGG-CM for 16 hrs (A) or a 1:10 dilution for varying times (B) and (C). Hsp protein expression was determined by Western blotting. As a positive control, cells were heat shocked for 23 minutes at 42° C. followed by a two hour recovery (HS). The effect of live bacteria (LGG bact.) is also shown (D). In this case, bacteria were left to co-incubate with YAMC cells for one hour, then washed off and cells were harvested 16 hours later. Images shown are representative of those of three separate experiments.

FIG. 15: LGG-CM requires only transient exposure to induce Hsps and rapidly activates MAP kinases YAMC cells were treated with vehicle control (MRS), or treated with 1:10 dilution of LGG-CM for varying times followed by removal and replacement with regular medium for 16 hours overnight (“O/N”). (A) Hsp induction was determined by Western blotting as described herein. Image shown is representative of three separate experiments. (B) YAMC cells were treated with 1:10 dilution of LGG-CM for 15 minutes and then MAP kinase activation was determined by Western blotting with specific antibodies to activated ERK1/2, p38, or SAPK/JNK. Samples were always analyzed for total expression of these kinases using antibodies which recognize both active and inactive forms. Phorbol 12,13 myristate acetate (PMA) was used as a positive control to activate ERK1/2 and anisomycin was used as a positive control to stimulate p38 and JNK. Image shown is representative of three separate experiments. (C) YAMC cells were treated with inhibitors of MAP kinases for 2 hours (p38 inhibitor SB203580 (20 μM), JNK inhibitor SP600125 (20 μM), ERK inhibitor PD98059 (50 μM)). Cells were then treated for 15 minutes with LGG-CM followed by removal and replacement with regular medium. Cells were harvested 4 hours later to analyze for Hsp72 induction by Western blotting as described herein. Densitometry for the corresponding lanes was determined using NIH Image J software and is indicated by the numbers below each lane of the Hsp72 autoradiogram. The inhibitory activity of the MAP kinase inhibitors used in (C) was tested and this is shown in panel (D). Image shown is representative of three separate experiments.

FIG. 16: Transient exposure to LGG-CM activates Akt kinase. YAMC cells were treated with LGG-CM at a concentration of 22.5% for 15 minutes, then media was removed and cells were harvested for varying times as indicated. NoTx=untreated cells, V-ctrl=cells treated with vehicle control. Akt kinase activation (P-Akt) was determined by Western blotting with specific antibodies to activated (phosphorylated) Akt. Samples were analyzed for total expression of Akt as well as for Hsc73 (loading control). TNFα (100 ng/ml) was used as a positive control to activate Akt. Akt was inhibited by pretreatment with LY294002 (50 μM) for 1 hour prior to LGG-CM or TNF treatment. Image shown is representative of three separate experiments.

FIG. 17: LGG-CM protects YAMC cells from monochloramine injury and oxidant-induced actin depolymerization. (A) YAMC cells were treated with MRS (Control) or treated with 1:10 dilution of LGG-CM for one hour followed by removal and recovery for 15 hours. Cells were labeled with ⁵¹Cr for 60 minutes and monochloramine (NH₂Cl)-induced release of Cr measured as described herein. Concentrations of NH₂Cl used are indicated. Data are means±SE for three separate experiments; in each experiment each point determined in duplicate. *p<0.05 compared with control by analysis of variance. (B) Cells were treated with PD98059 (50 μM), SB203580 (20 μM), or SP600125 (20 μM) for two hours prior to addition of LGG-CM. Cells were then treated with LGG-CM for one hour, after which media was replaced with fresh media (i.e., no inhibitors or LGG-CM) and cells were returned to the incubator overnight before chromium loading and injury with 0.6 mM NH₂Cl as described herein. Data are means±SE for four separate experiments; in each experiment each point was determined in duplicate. ++p<0.001, and *p<0.05 compared to NH₂Cl-treated control by analysis of variance using a Bonferroni correction. (C) siRNA was used to knock down or inhibit Hsp25 and Hsp72 expression as described herein; cells were then treated with LGG-CM for one hour the day prior to chromium loading and injury with 0.6 mM NH₂Cl as above. Data are means +SE for four separate experiments; in each experiment each point was determined in duplicate. *p<0.01 when compared with NH₂Cl-treated control and +p<0.05 when compared to Hsp72 siRNA by analysis of variance using the Bonferroni method. Western blot analysis was performed to confirm silencing of Hsp expression, as shown. (D) YAMC cells were untreated or treated with LGG-CM as above and stimulated with 0.6mM NH₂Cl for 60 minutes and cells were harvested for assay of actin distribution in filamentous (F) and globular (G) pools as described herein. Images shown are representative of three separate experiments.

FIG. 18: Characterization of LGG-CM active factor. (A) Molecular mass determination. YAMC cells were untreated (NOTX), or treated with the retentate, filtrate, or a combination of both (R+F) from a 10 kDa molecular mass cutoff filter. Heat shock (HS) at 42° C. for 23 minutes followed by a two-hour recovery was used as a positive control. Image shown is representative of three separate experiments. (B) YAMC cells were treated with vehicle control (MRS), treated with a 1:10 dilution of LGG-CM (alone or filtered (F)), or treated with various proteases and then filtered to remove residual proteases prior to administration to YAMC cells. Proteases used to treat LGG-CM or MRS broth control (pepsin, trypsin, Proteinase K) are as indicated (final concentration of 50 μg/ml for each). Hsp induction was determined by Western blotting as described herein. B-actin serves as a loading control. Images shown are representative of those of three separate experiments. (C) Heat stability. YAMC cells were untreated (NOTX) or treated with LGG-CM untreated (LGG) or boiled (LGG boiled) and Hsp induction was determined by Western blotting. Heat shock (HS) at 42° C. for 23 minutes followed by 2 hour recovery was used as a positive control. Image shown is representative of three separate experiments. (D) YAMC cells were either mock treated (first column, 0 ng/ml) or treated with DNA isolated from LGG bacteria at varying concentrations and then tested for Hsp induction using an Hsp72 ELISA assay. Herring sperm DNA was used as a negative DNA control; the positive control from heat shock (HS) is also shown (n=3).

FIG. 19: The bioactive factor(s) in LGG-CM possess unusual pH properties. (A) LGG-CM with different pH treatments (as indicated) were used to treat YAMC cells immediately after pH manipulation and Hsp induction was determined by Western blotting as described herein. MRS broth controls were subjected to the same pH treatments to ensure that the pH change itself was not causing the induction of Hsps (lanes 6 and 7). Hsc73 serves as a loading control. (B) LGG-CM was treated as in (A) except that after its pH was returned to pH 4, it was left to incubate overnight (lane 4) and then used to treat YAMC cells as in (A) to determine whether the Hsp-inducing activity of LGG-CM could be restored over time at pH 4. NOTX=untreated control cells (lane 1). HS=heat-shocked cells (lane 5). Images shown are representative of three separate experiments.

### DETAILED DESCRIPTION OF THE INVENTION

A. GENERAL DESCRIPTION

Probiotics are living organisms, mostly found in food supplements, which provide health benefits beyond their nutritive value. Probiotic compounds according to the invention are expected to have multiple beneficial effects on the host. Soluble factors produced by a common probiotic, Lactobacillus GG, act on epithelial cells to produce a time- and concentration-dependent induction of the cytoprotective heat shock proteins Hsp25 and Hsp72. The soluble factors produced by LGG are sufficient for Hsp induction and live bacteria are not required. Although the actual appearance of Hsp protein takes hours, washout experiments in which cells were exposed to LGG-CM for only a few minutes show that the time required to initiate the signal to the epithelial cells for upregulation of heat shock proteins is very short, indicating that signal transduction pathways play a role in transmitting the initiating signal from LGG-CM to the epithelial cell. Many protein kinases are known to be activated during the stress response, and it was confirmed that LGG-CM exposure activates a number of MAP kinases. Although there is a baseline level of activated ERK1/2 in YAMC (young adult mouse colon) cells, LGG-CM pretreatment activates ERK1/2 as effectively as the phorbol ester PMA and also activates p38 and JNK. Treatment of cells with inhibitors of p38 and JNK prior to LGG-CM exposure inhibited Hsp72 induction by LGG-CM with no effect on Hsc73 expression. This indicates that these two MAP kinases play roles in transmitting the cellular signal required to initiate the expression of inducible Hsps triggered by exposure to LGG-CM. Other studies have revealed that p38 and JNK act through a common pathway that is distinct from ERK1/2 (Liu et al., 1996). These results demonstrate that LGG-CM affects signal transduction in epithelial cells and, hence, indicates that at least one rapid signaling for the induction of Hsp production is initiated through at least one signal transduction pathway.

Conditioned media (throughout the disclosure, “medium” and “media” are used interchangeably and may be used in the singular or plural number which will be apparent from context) from the probiotic Lactobacillus GG (LGG-CM) induces heat shock protein expression in intestinal epithelial cells. Both Hsp25 and Hsp72 are induced by LGG-CM in a time- and concentration-dependent manner. Moreover, these effects are mediated by a small molecular weight peptide that is both acid- and heat-stable. DNA microarray experiments demonstrate that in epithelial cells Hsp72 (Hsp70) is one of the most highly upregulated genes in response to LGG-CM treatment. Real-time PCR and electrophoretic mobility shift assays confirm that regulation of Hsp induction is at least in part transcriptional in nature and involves transcription factor HSF-1. Even though Hsps are not induced for hours post-exposure, transient exposure to LGG-CM is sufficient to initiate the signal for Hsp induction and, given the rapidity of the response, such times suggest that signal transduction pathways may be involved. Experiments confirm that LGG-CM modulates the activity of certain signaling pathways in intestinal epithelial cells by activating MAP kinases. Inhibitors of p38 and JNK block the expression of Hsp72 normally induced by LGG-CM. Functional studies indicate that LGG-CM treatment of gut epithelial cells protects them from oxidant stress and preserves cytoskeletal integrity (Mounier et al., 2002). By inducing the expression of cytoprotective Hsps in gut epithelial cells, and/or by activating signal transduction pathways, the isolated peptide product secreted by LGG contributes to the beneficial clinical effects of this peptide.

The invention demonstrates that bioactive compounds can be isolated from Lactobacillus GG (LGG), a probiotic microbe. The use of bacteria-free, probiotic-derived compounds instead of live bacteria provides a safety advantage over the use of live bacteria. In addition, the clinical efficacy of isolated compounds is likely to be more consistent than for probiotics, which depend on the ability to establish and maintain bacterial colonization.

The factor present in LGG-CM responsible for heat shock protein induction is a small molecular weight peptide which is surprisingly acid- and heat-stable. These properties may provide the resilience needed for such secreted factors to survive the hostile environment of the gut on their transit through the GI tract. It is also interesting to note that the physicochemical environment in the middle of the gut lumen is quite different from that found at the epithelial surface, which tends to be more acidic (Rechkemmer et al., 1986). This acid microclimate is thought to play an important role in functions such as membrane transport, drug uptake, and nutrient absorption (Sanderson, 1999). It has been shown that the acid microclimate has a direct effect on the transport of certain dipeptides into the intestinal epithelial cell (Lister et al., 1995). If the bioactive peptide in LGG-CM acts through a receptor-mediated pathway, its unusual acid-stable property may play an important role in its ability to bind to receptors on the surface of the epithelial cell and initiate induction of cytoprotective Hsps.

Thus, the compounds of the invention will provide novel therapies for the treatment of inflammatory disorders, such as IBD, that are superior to those currently available in the art. In one aspect, the invention provides a composition comprising an isolated cytoprotective compound isolatable or derivable from Lactobacillus GG. In addition, the invention provides methods for preventing, treating or ameliorating at least one known symptom of a patient with an inflammatory disorder comprising administering to the patient an effective dose or amount of a cytoprotective compound derived from Lactobacillus GG. In other aspects, the invention provides methods for isolating and characterizing compounds derived from Lactobacillus GG-conditioned media that have cytoprotective properties.

B. IDENTIFICATION AND CHARACTERIZATION OF CYTOPROTECTIVE COMPOUNDS

The invention provides methods of identifying and characterizing compounds derived from bacterial cultures that have cytoprotective properties. The invention also provides cytoprotective compounds isolatable from probiotic organisms, as well as compositions and methods useful in treating, preventing or ameliorating at least one symptom of an inflammatory diseases.

1. Isolation of Cytoprotective Compounds

Any bacterial strain or probiotic formulation is suitable for screening for cytoprotective compounds. Preferably, the bacteria are non-pathogenic, enteric bacteria. In the exemplary embodiments disclosed herein, the bacterium is Lactobacillus GG. Methods of bacterial culture are well known to those of skill in the art. MRS broth is commonly used for the isolation and cultivation of Lactobacillus species. For example, Lactobacillus GG grows readily in MRS broth under microaerophilic conditions in 5% CO₂ at 37° C. Another example of media used for the cultivation of Lactobacillus species is tomato juice broth.

The cytoprotective compounds were determined to be soluble factors found in Lactobacillus GG-conditioned media. To facilitate the identification and characterization of these compounds it is preferable to remove the bacterial cells from the media. One of skill in the art would be familiar with methods of separating cells from the soluble factors in the media. For example, the cells may be removed by centrifugation, filtration or a combination of both techniques. Following the removal of the bacterial cells, the “conditioned medium” is then used as the source from which isolated cytoprotective compounds may be further purified and characterized.

(a) Other Separation Techniques

Other separation techniques known to those of skill in the art are also expected to be useful in fractionating conditioned media, thereby isolating probiotic factor(s) (i.e., bioactive agents). High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Fast protein liquid chromatography (FPLC) is a technique commonly used for protein separation. FPLC is basically a form of HPLC that runs under low pressure, with a “resin” made from an inert substance like cellulose or dextran, with chemical side groups attached to it to give it specific binding properties. The side chains determine the type of chromatography. For example, hydrophobic LC separates proteins by the amount of hydrophobic amino acids they contain; ion exchange LC separates proteins by the number and type of charged amino acids; ligand/affinity LC separates proteins by their specificity to certain substrates, dyes or antibodies; and size exclusion LC (or gel filtrations separates proteins by their size.

Gel filtration chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is size. Hence, molecules are eluted from the column in order of decreasing size, so long as the shape is relatively constant. Gel chromatography is unsurpassed for separating molecules of different size because separation is independent of all other factors, such as pH, ionic strength, temperature, and the like. There also is virtually no adsorption, less zone spreading and the elution volume is related in a simple manner to molecular weight.

Additionally, the conditioned media may be passed through filters with specific molecular weight cutoffs. For example, some fractions of the invention were prepared through Centricon filters with specific molecular weight cutoffs.

Separation techniques based on charge are also contemplated. One such technique is ion exchange chromatography. With ion exchange chromatography, the sample is reversibly bound to a charged matrix. Matrices containing diethyl aminoethyl (DEAE) and carboxymethyl (CM) cellulose are commonly used. Desorption is then brought about by increasing the salt concentration or by altering the pH of the mobile phase. Based on chromatography data disclosed herein establishing that the cytoprotective factor in LGG-CM is stable at low pH, its isoelectric point is expected to be low and, therefore, the protein should be negatively charged, rendering it a good candidate for charge-based separation techniques. One approach to isolation thus involves subjecting the conditioned media to anion exchange chromatography on appropriately derivatized (e.g., introduction of positively charged functional groups at the desired pH) Mono S columns, as would be known in the art. To expedite the isolation, these columns could be used in an FPLC system.

Another technique known to those skilled in the art for separating compounds based on charge is IEF (isoelectric focusing). One approach that typically utilizes IEF is two-dimensional electrophoresis. Two-dimensional gel electrophoresis can be performed using methods known in the art (See, e.g., U.S. Pat. Nos. 5,534,121 and 6,398,933). Typically, proteins in a sample are separated first by isoelectric focusing, during which proteins in a sample are separated by pI in a pH gradient until they reach a spot where their net charge is zero (i.e., isoelectric point or pI). This first separation step results in a one-dimensional array of proteins. The proteins in the one-dimensional array are further separated in the second dimension using a technique generally distinct from that used in the first separation step. For example, in the second dimension, proteins separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of the protein.

Proteins in the two-dimensional array can be detected using any suitable methods known in the art. Staining of proteins can be accomplished with colorimetric dyes (e.g., Coomassie), silver staining and fluorescent staining (e.g., Ruby Red). As is known to one of ordinary skill in the art, protein spots can be excised from the gel and analyzed by gas-phase ion spectrometry. Alternatively, the proteins can be transferred from the gel to an inert membrane, e.g., by applying an electric field, and then analyzed by gas-phase ion spectrometry.

The protein separation techniques described above may be used alone or in any combination. During the course of purification or isolation it may be desirable to assay the fractions in order to follow those fractions that retain cytoprotective activity. For example, the media or fraction may be screened for the ability to induce the expression of at least one cytoprotective heat shock protein. These assays are described in more detail below. Preparations that have biological activity may be frozen in aliquots to be used later for identification, purification, and future production of anti-inflammatory and cytoprotective compounds.

2. Identification of Cytoprotective Compounds

The cytoprotective compounds of the invention are identified by methods of protein identification known to those of skill in the art.

For example, the conditioned media would first be fractionated, and the fractions tested for bioactivity (e.g., the ability to induce heat shock protein expression). The purity of fractions retaining activity is determinable, for example, by PAGE followed by silver-staining analysis. If the active components have been resolved to homogeneity, the protein may be analyzed in at least two ways. First, amino acid analysis can be performed by subjecting the samples to 6N hydrolysis with HCl in an evacuated tube for 24 hours followed by HPLC-Mass sequencing to identify all the individual amino acids that comprise the peptide of interest. Second, internal amino acid sequences can be determined from readily identified tryptic fragments. The isolated protein is typically concentrated and then treated with trypsin. The fragments are dried and then resolved on a C18 reverse-phase HPLC and the amino acid sequence of those peaks determined by matrix-assisted laser desorption ionization—time of flight mass spectrometry (MALDI-TOF). The amino acid sequences are then compared with one or more known sequences, e.g., sequences found in any known database (SwissProt, Genbank) to identify the protein or peptide of interest.

3. Characterization of Cytoprotective Compounds

The compounds found in LGG-conditioned media can be assayed for cytoprotective properties using the methods described here or known in the art. Indicators of cytoprotective activity include, for example, the ability to induce the expression of heat shock proteins and the ability to reduce inflammation in a subject with an inflammatory disorder.

(a) Heat Shock Proteins

The data disclosed herein demonstrates that LGG-conditioned media induces the expression of heat shock proteins, specifically at least Hsp72 and Hsp25. Heat shock proteins are a family of proteins that protect a cell against environmental stressors. Hsp72 binds and stabilizes critical cellular proteins, preventing their denaturation. It also has anti-apoptotic actions through preservation of mitochondrial integrity, inhibition of cytochrome C leakage, and blockade of caspase 8 activity (Liu et al., 2003). Hsp25/27 is an actin-stabilizing agent and preserves cytoskeletal and tight junction functions. The ability to induce heat shock protein expression indicates that the active compound(s) in LGG-conditioned media are cytoprotective.

Methods of analyzing the induction of heat shock proteins are known to those of skill in the art. For example, the induction of Hsp72 and Hsp25 can be performed by standard Western blot analysis using monoclonal antibodies to the specific isoforms (Stressgen). Immunoblots for the constitutive heat shock cognates, Hsp60 and Hsc73, can be performed to check the specificity of response and ensure equal loading of lanes (the expression of these proteins usually remains constant). In addition, antibodies can be used to detect the expression of heat shock proteins by immunofluorescence and ELISA.

Other methods of analyzing the induction of heat shock proteins include assaying Hsp mRNA levels using, for example, RT-PCR, genomic microarrays, and real-time PCR. Another approach for analyzing the induction of heat shock proteins is the use of electrophoretic mobility shift assays to look at binding of the transcription factor HSF-1. In addition, HSE-luciferase reporter assays can be employed to measure the activity of the transcription factor HSF-1.

(b) Animal Models

The characterization of the compounds of the invention may involve the use of various animal models, including transgenic animals that have been engineered to have specific defects, or carry markers that can be used to measure the ability of a candidate substance to reach and affect different cells within the organism. Due to their size, ease of handling, information on their physiology and genetic make-up, and art-recognized validity as a model for human inflammation, mice are a preferred animal model, especially for studies of transgenics. However, other animals are suitable as well, including rats, rabbits, hamsters, guinea pigs, gerbils, woodchucks, cats, dogs, sheep, goats, pigs, cows, horses, and monkeys (including chimps, gibbons and baboons). Assays may be conducted using an animal model derived from any of these species.

Some examples of mouse models for colitis include the DSS-induced colitis model, the IL-10 knockout mouse, the A20 knockout mouse, the TNBS-induced colitis model, the IL-2 knockout mouse, the TCRalpha receptor knockout mouse, and the E-cadherin knockout mouse.

Treatment of animals with test, or candidate, compounds will involve the administration of the compound, in an appropriate form, to the animal. Any animal model of inflammatory disease known to those of skill in the art is contemplated. Administration is by any route that could be utilized for clinical or non-clinical purposes. For example, the compound may be delivered by gavage or by rectal administration. In addition, the protective effects of a compound are assayed by administering a compound before inducing, e.g., colitis in the animal model. Alternatively, the therapeutic effect of a compound is assayed by administering the compound after inducing, e.g., colitis in the animal model.

Determining the effectiveness of a compound in vivo involves a variety of different criteria. One of ordinary skill in the art would be familiar with the wide range of techniques available for assaying for inflammation in a subject, whether that subject is an animal or a human subject. For example, inflammation is measured by histological assessment and grading of the severity of colitis. Other methods for assaying inflammation in a subject include, for example, measuring myeloperoxidase (MPO) activity, transport activity, villin expression, or transcutaneous electrical resistance (TER).

The effectiveness of a compound may also be assayed using tests that assess cell proliferation. For example, cell proliferation may be assayed by measuring 5-bromo-2′-deoxyuridine (BrdU) uptake. Yet another approach to determining the effectiveness of a compound is to assess the degree of apoptosis. Methods for studying apoptosis are well known in the art and include, for example, the TUNEL assay.

In addition, measuring toxicity and dose response can be performed in animals in a more meaningful fashion than in in vitro or in cyto assays and are routine procedures in the art.

### C. PHARMACEUTICAL COMPOSITIONS

Compositions of the invention comprise an effective amount of a cytoprotective compound, which may be dissolved and/or dispersed in a pharmaceutically acceptable carrier and/or aqueous medium.

The cytoprotective compounds of the invention are delivered by any method known to those of skill in the art (see for example, “Remington's Pharmaceutical Sciences” 15th Edition). For example, the pharmaceutical compositions may be delivered orally, rectally, parenterally, or topically.

Solutions comprising the compounds of the invention may be prepared in water suitably mixed with a surfactant, such as polyethylene glycol (PEG) or hydroxypropylcellulose. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. The form should usually be sterile and must be fluid to the extent that operable syringability exists. It must be stable under the conditions of manufacture and storage and must be preserved against the contaminating action of microorganisms, such as bacteria and fungi.

For parenteral administration in an aqueous solution, for example, the solution is suitably buffered if necessary and the liquid diluent first rendered isotonic with sufficient saline or glucose. These particular aqueous solutions are especially suitable for intravenous, intramuscular, subcutaneous, intratumoral, and intraperitoneal administration. In this connection, sterile aqueous media that can be employed will be known to those of skill in the art in light of the present disclosure.

A suppository may also be used. Suppositories are solid, including gel, dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of, e.g., 0.5% to 10%, preferably 1%-2%. The pharmaceutical compositions of the invention may also be delivered by enema.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard- and/or soft-shell gelatin capsules, and/or they may be compressed into tablets, and/or they may be incorporated directly into the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, preferably between 25-60%. The amount of active compounds in such therapeutically useful compositions is such that a suitable dosage, as known or determinable in the art, will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the unit dosage form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent, methyl and/or propylparabens as preservatives, a dye and/or flavoring, such as cherry and/or orange flavor.

Topical formulations include creams, ointments, jellies, gels, epidermal solutions or suspensions, and the like, containing the active compound.

For human administration, preparations should meet sterility, pyrogenicity, general safety and purity standards as required by FDA Office of Biologics standards.

The phrases “pharmaceutically acceptable” and “pharmacologically acceptable” refers to molecular entities and compositions that do not produce an allergic or similar untoward reaction when administered to a human.

The dosage of the cytoprotective compound(s) and dosage schedule may be varied on a subject-by-subject basis, taking into account, for example, factors such as the weight and age of the subject, the type of disease being treated, the severity of the disease condition, previous or concurrent therapeutic interventions, the manner of administration and the like, which can be readily determined by one of ordinary skill in the art.

Administration is in any manner compatible with the dosage formulation, and in such amount as will be therapeutically effective. The quantity to be administered depends on the subject to be treated. Precise amounts of an active ingredient that is required to be administered depend on the judgment of the practitioner.

### D. EXAMPLES

The following examples are included to demonstrate preferred embodiments of the invention. It will be appreciated by those of skill in the art that the techniques disclosed in the examples which follow represent techniques disclosed herein to function well in the practice of the invention. However, those of skill in the art should, in light of the present disclosure, appreciate that many changes can be made in the specific embodiments which are disclosed and still obtain a like or similar result without departing from the spirit and scope of the invention.

All experiments reported in the following examples were repeated a minimum of three to six times each. All numerical values are expressed as mean±standard error of the mean. Where multiple comparisons were made, ANOVA analysis using Bonferroni's correction was used to assess the significance of differences between groups. P<0.05 was considered statistically significant.

### Example 1

Cultures

Tissue Culture. YAMC (young adult mouse colon) cells are a conditionally immortalized mouse colonic intestinal epithelial cell line derived from the Immortimouse Whitehead et al., 1993). These cells express a transgene of a temperature-sensitive SV40 large T antigen (tsA58) under control of an interferon-gamma sensitive portion of the MHC class II promoter (Whitehead et al., 1993, incorporated herein by reference). This special feature allows YAMC cells to be cultured under non-permissive (non-transformed) conditions at 37° C. in the absence of interferon-gamma (IFN-γ). YAMC cells were maintained under permissive conditions (33° C.) in RPMI 1640 medium with 5% (vol/vol) fetal bovine serum, 5 U/ml murine IFN-γ (GibcoBRL, Grand Island, N.Y.), 50 μg/ml streptomycin, and 50 U/ml penicillin, supplemented with ITS-X Premix (Collaborative Biomedical Products, Bedford, Mass.).

Under non-permissive (non-transformed) conditions at 37° C. in the absence of IFN-γ, these cells undergo differentiation and develop mature epithelial cell functions and properties including tight junction formation, polarity, microvillar apical membranes, and transport functions.

Prior to each study, cells were plated at a density of 2×10⁵ per 60 mm tissue culture dish. For RNA preparation, cells were plated at a density of 7.5×10⁵ cells per p100 mm dish. After 24 hours of growth at 33° C., the medium was replaced with IFN-free media and cells were moved to 37° C. (non-permissive conditions) for 24 hours to allow development of the differentiated colonocyte phenotype. Cells were treated with LGG-conditioned media (1:10 dilution, or 600 μl) overnight, and then harvested the following day. Heat shock controls were heat-shocked at 42° C. for 23 minutes and then left at 37° C. for 2 hours before harvest.

Bacterial Culture and Preparation of LGG-conditioned Medium (LGG-CM).

The probiotic, Lactobacillus GG (ATCC 53103), was grown to a concentration of approximately 2×10⁹ CFU/ml (as determined by colony counts) in MRS broth (per liter of broth contains: 10 g Bacto Proteose Peptone #3, 10 g Bacto Beef Extract, 5 g Bacto Yeast Extract, 20 g Bacto Dextrose, 1.0 g Polysorbate 80, 2.0 g Ammonium Citrate, 5.0 g Sodium Acetate, 0.1 g Magnesium Sulfate, 0.05 g Manganese Sulfate, 2.0 g Dipotassium Phosphate) for 16 hours or in HBSS (Hank's Balanced Salt Solution, In Vitrogen, Carlsbad, Calif.), then centrifuged at low speed in a tabletop Sorvall centrifuge (3000×g) at 4° C. for 10 minutes. The supernatant (conditioned media) was filtered through a 0.22 micron low protein-binding Millex filter (Millipore, Billerica, Mass.) to sterilize and remove all bacterial cells. Aliquots of LGG-conditioned media were stored in sterile microcentrifuge tubes at −80° C. until further use.

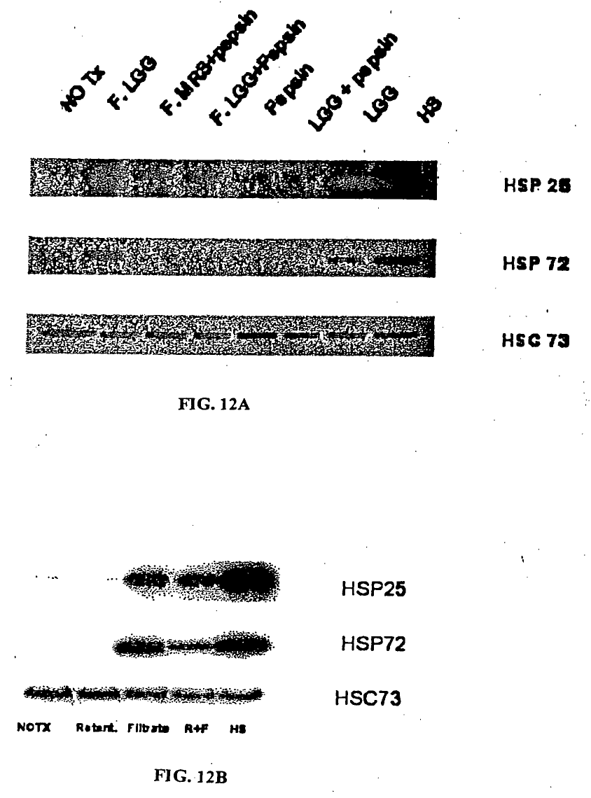

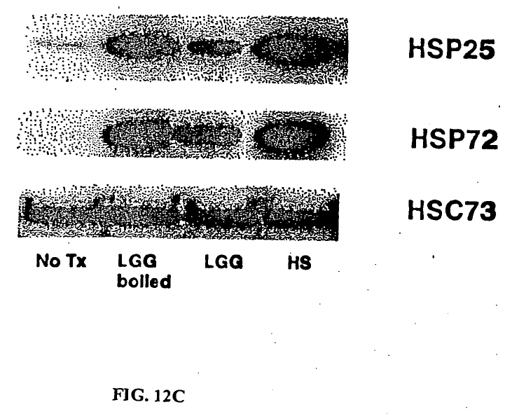

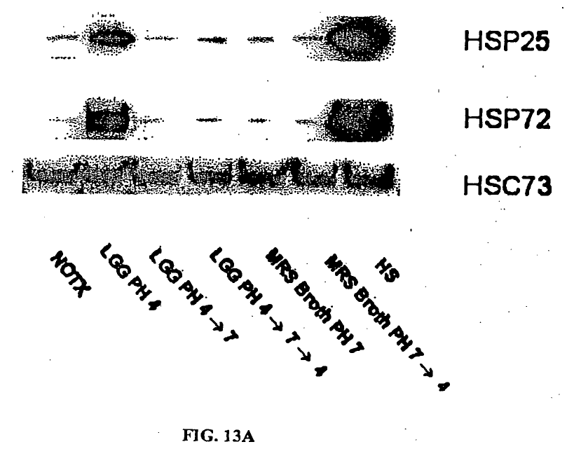

The LGG-CM was subjected to selective ultrafiltration to determine the molecular mass of the active factor. The filtrate (containing molecules of less than 10 kDa) and the retentate (containing molecules larger than 10 kDa) or both together were then used to treat YAMC cells and immunoblots for Hsp25 and Hsp72 were performed. Only the filtrate (FIG. 12, lane 3) or both fractions administered together (FIG. 12, lane 4, R+F) induce Hsp expression in YAMC cells, indicating that the bioactive factor(s) is a protein or peptide of small molecular mass less than 10 kDa. Further characterization of the active peptide revealed that it is heat stable, still retaining activity even after boiling for 10 minutes in a sand bath, followed by cooling to room temperature before use (FIG. 12C, compare lanes 2 and 3).

For the protease experiments, LGG-CM was prepared as above and then, because pepsin is active at acidic pH, was treated directly with pepsin (#P7012 Sigma Chemical Co., St. Louis, Mo.) at a working concentration of 0.01-0.05 mg/ml for 90 minutes at room temperature, then subjected to sizing filtration to remove the pepsin (molecular weight of 34.7 kDa) using a 10 kDa cutoff centricon spin column (Millipore, Bedford, Mass.) prior to treating the cells. For the trypsin and proteinase K treatments, these enzymes are not active at acidic pH and so it was necessary to first adjust the LGG-CM pH from 4.0 to 8.0 using concentrated NaOH, then LGG-CM was treated with either trypsin (#115400-054 GIBCO) or proteinase K (#BP1700-100 Fisher Biotech) at a final concentration of 50 [g/ml for 90 minutes at 37° C. As the LGG-CM regains its activity when the pH is returned to 4.0, the pH of the treated LGG-CM was readjusted to 4.0 by concentrated HCl, then subjected to sizing separation using 10 kDa spin columns as described above to remove the trypsin (24 kDa) or proteinase K (28.9 kDa) prior to treating the cells. YAMC cells were treated with the LGG-CM filtrate at 1:10 concentration for 16 hours and cells were harvested for Western blot analysis as described herein.

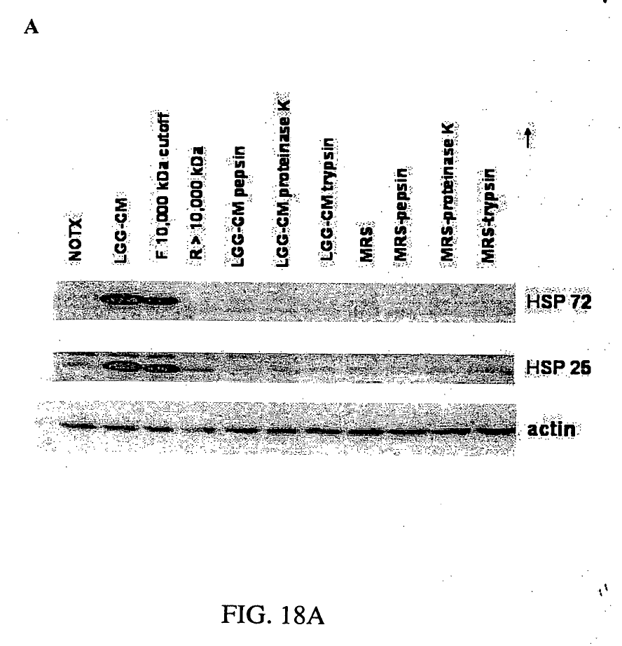

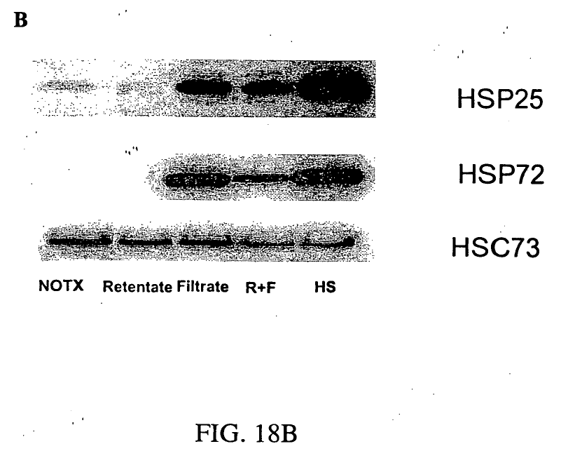

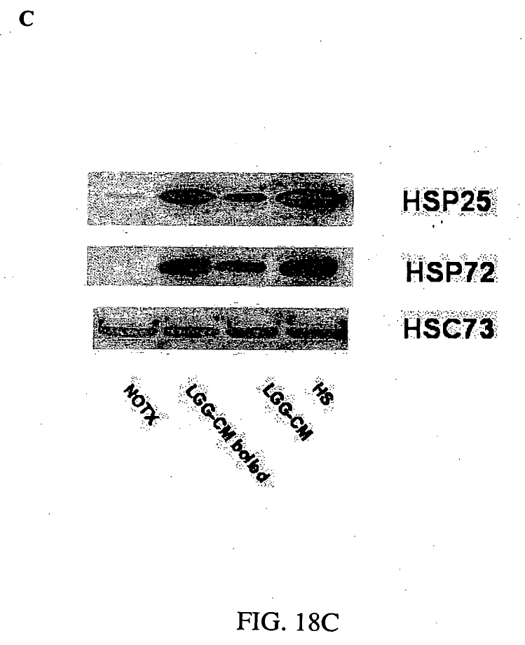

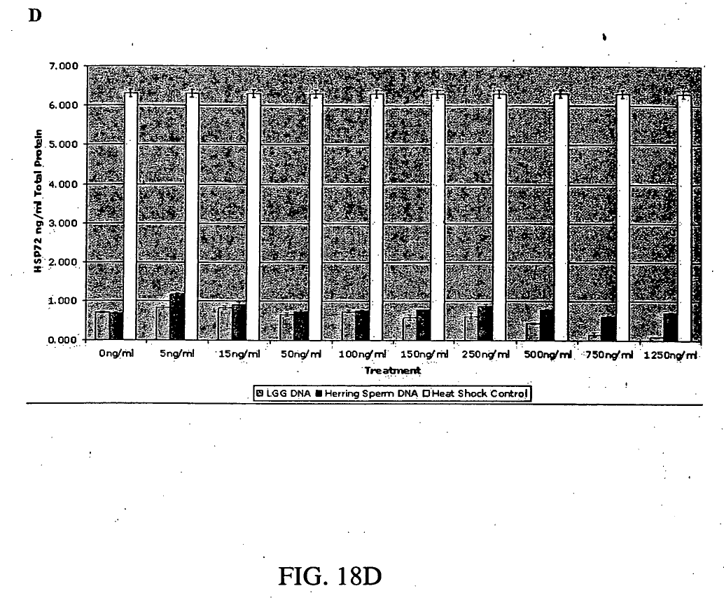

As the LGG-CM bioactivity is located in the small molecular weight fraction, the possibility that the bioactive factor was DNA was explored. DNA was isolated from LGG bacteria and epithelial cells were treated with LGG DNA over a range of several concentrations and then screened for induction of Hsp72 expression by ELISA (FIG. 18D). When compared to untreated control (see FIG. 18D, first column, marked 0 ng/ml), no induction was seen over any of the concentrations tested, suggesting that the bioactive factor is not comprised of DNA.

For the DNA experiments, DNA was isolated from LGG bacteria using a method modified from a protocol originally used to isolate DNA from Listeria, another Gram-positive bacillus bacteria (Flamm et al., 1984). Briefly, 10 ml of LGG was grown overnight in MRS medium using the same method as described above, then bacteria were pelleted and resuspended in 1.0 ml lysozyme buffer (2.5 mg/ml lysozyme, 10 mM Tris, 20% sucrose), and incubated at 37° C. for 45 minutes. To this mixture, 9 ml of pronase lysis buffer (500 μg pronase, 1% SDS, 1 mM EDTA, 10 mM Tris) was added and incubated for an additional hour at 37° C. DNAzol (In Vitrogen, Carlsbad, Calif.) was then added according to the manufacturer's instructions, and the solution was gently mixed, followed by centrifugation at 10,000×g at 4° C. for 10 minutes; the supernatant was removed to a fresh tube. To this solution, 100% ethanol was added to allow precipitation of the DNA until the solution turned cloudy (about 5 ml), and this was left to incubate at room temperature for 5 minutes. The solution was then centrifuged to pellet the DNA according to the manufacturer's instructions, washed in 70% ethanol and resuspended in water. DNA concentration was determined using absorbance at 260 nm using a spectrophotometer (MiraiBio, Alameda, Calif.) and the newly isolated DNA was used immediately to treat the intestinal epithelial cells at varying concentrations. Herring sperm DNA was used as a negative DNA control. Cells were harvested the following day and processed for analysis of Hsp72 induction by ELISA (see Example 11).

To test the possibility that the bioactive factor may be a protein or peptide, LGG-CM was treated with several different proteases and then tested for bioactivity. First, the protease pepsin was chosen because LGG-CM has a pH of approximately 4 and pepsin is the protease with best activity at this pH. LGG-CM was first treated with pepsin and then filtered through a 10 kDa sizing column to remove any residual pepsin. Pepsin treatment destroys the bioactivity of LGG-CM and no induction of Hsps is seen in the YAMC cells, indicating that the bioactive factor is a protein or peptide (FIG. 18A). To confirm these findings, LGG-CM was treated with two other proteases (trypsin, proteinase K), and the proteases were then filtered out as described above. Again, Hsp-inducing activities were abolished after protease treatment, providing strong supportive evidence that the bioactive factor found in LGG-CM is a peptide or protein.

### Example 2

LGG-CM Induces Expression of Hsp25 and Hsp72 in Intestinal Epithelial Cells—Western Analyses

Intestinal epithelial cells were treated with conditioned media from the probiotic LGG and then assayed for inducible heat shock protein expression. For Western blot analyses of expression, cells were washed twice and then scraped in ice-cold PBS (137 mM NaCl, 2.7 mM KCl, 8 mM Na₂HPO₄, pH 7.4). Cells were pelleted (14,000×g for 20 seconds at room temperature), then resuspended in ice-cold lysis buffer (10 mM Tris, pH 7.4, 5 mM MgCl₂, 50 U/ml each of DNAse and RNAse, plus complete protease inhibitor cocktail (Roche Molecular Biochemicals, Indianapolis, Ind.)). Protein concentrations were determined using the bicinchoninic acid procedure (Smith et al., 1985). Samples were heated to 75° C. for 5 minutes after addition of 3× Laemmli Stop buffer, then stored at −80° C. until use.

Twenty micrograms of protein per lane were resolved on 12.5% SDS-PAGE. Samples were transferred in 1× Towbin buffer (25 mM Tris, 192 mM glycine, pH 8.8, 15% vol/vol methanol) onto PVDF membranes (Perkin-Elmer NEN, Boston, Mass.) as previously described (Kojima et al., 2003). Membranes were blocked in 5% (wt/vol) non-fat milk in TBS-Tween (Tris-buffered saline (150 mM NaCl, 5 mM KCl, 10 mM Tris, pH 7.4) with 0.01% (vol/vol) Tween 20) for one hour at room temperature. Primary monoclonal antibody, i.e., a specific anti-Hsp25 antibody (SPA801, Stressgen, Victoria, BC, Canada), anti-Hsp72 antibody (SPA 810, Stressgen), or either anti-Hsc 73 antibody (SPA 815, Stressgen) or actin (#AANO1, Cytoskeleton, Denver, Colo.), was added to TBS-Tween and incubated overnight at 4° C. The anti-Hsc 73 antibody and the actin served as controls for protein loading. Blots were washed five times in TBS-Tween before incubation with secondary antibody. Membranes were incubated with secondary antibodies conjugated to horseradish peroxidase (Jackson Immunoresearch Labs, Inc., Fort Washington, Pa.) for 1 hour at room temperature, then washed five times in TBS-Tween followed by a final wash in TBS (no Tween). Membranes were then treated with an enhanced chemiluminescence system ECL reagent (Supersignal, Pierce, Rockford, Ill.) and developed as per the manufacturer's instructions.

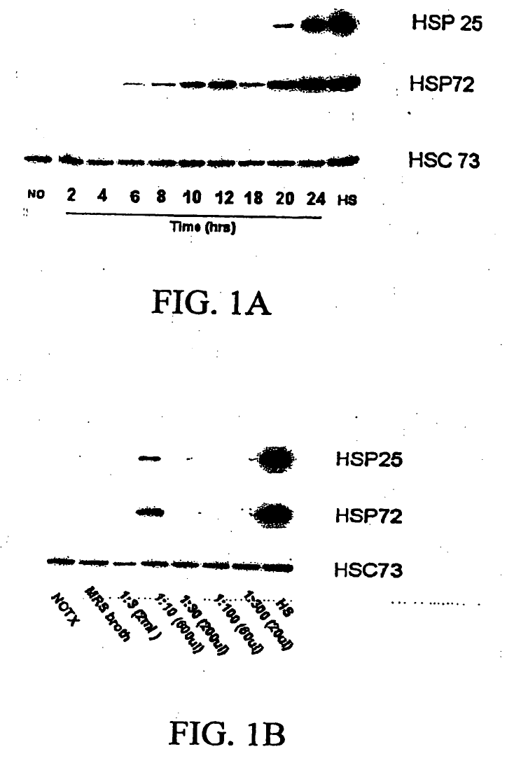

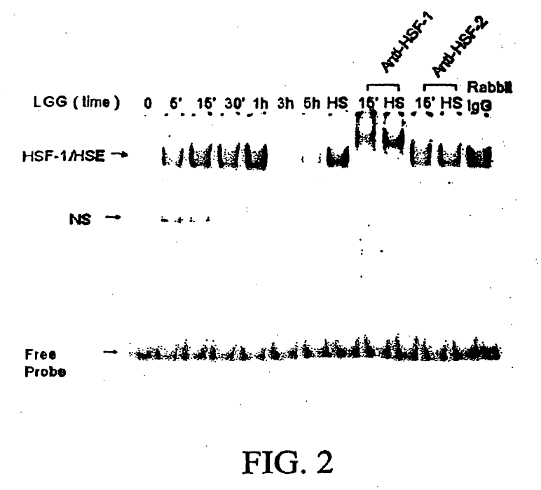

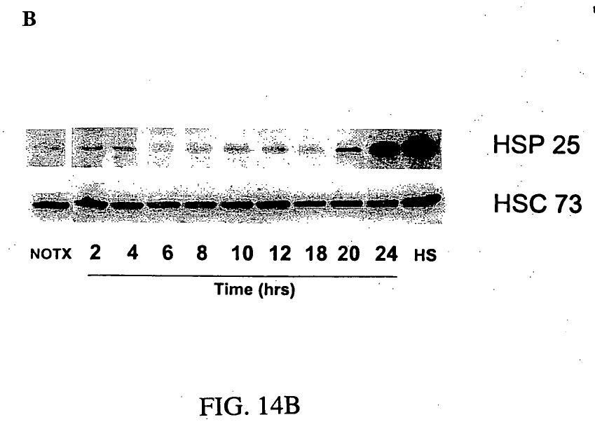

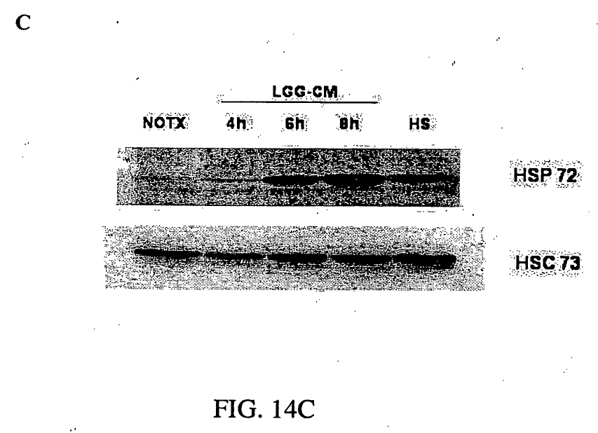

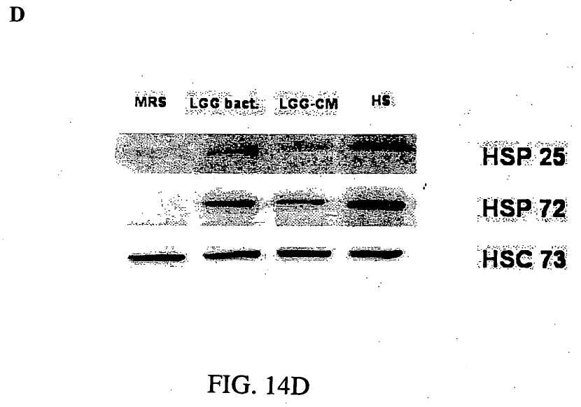

Conditioned medium from the probiotic LGG induced heat shock protein Hsp25 and Hsp72 expression in cultured murine colonic YAMC cells in a time-dependent manner, with the expression of Hsp25 beginning after 18-20 hours (FIG. 14B) and Hsp72 being expressed somewhat earlier, first appearing at 4-6 hours (FIG. 14C). During the course of this treatment, expression of the constitutively expressed housekeeping gene hsc73 did not change, indicating that the effect of LGG-CM is specific to inducible forms of heat shock protein. In addition, the epithelial cells responded to the LGG-CM in a concentration-dependent manner, with the most robust response being observed with a 1:10 dilution (FIG. 1B and FIG. 14A). The Hsp-inducing effects of live LGG bacteria are also shown (FIG. 14D). No heat shock response was observed in untreated cells (NOTX) (FIG. 14B, first lane; see also FIG. 1B) or in cells contacted with unconditioned MRS broth (FIG. 1B, second lane, and FIG. 14A, second lane).

Unlike the rapid response seen with thermal stress, which induces heat shock protein within a matter of two hours in YAMC cells, the response to LGG-CM took considerably longer. Thus, the mechanisms underlying Hsp induction by LGG-CM and thermal stress differed.

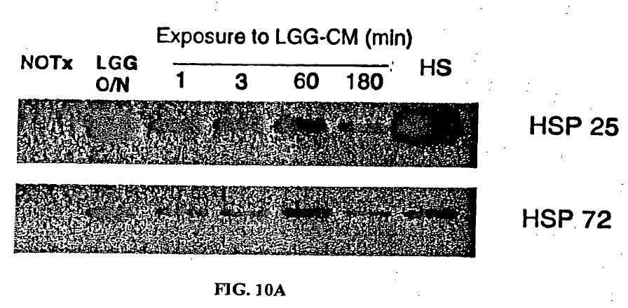

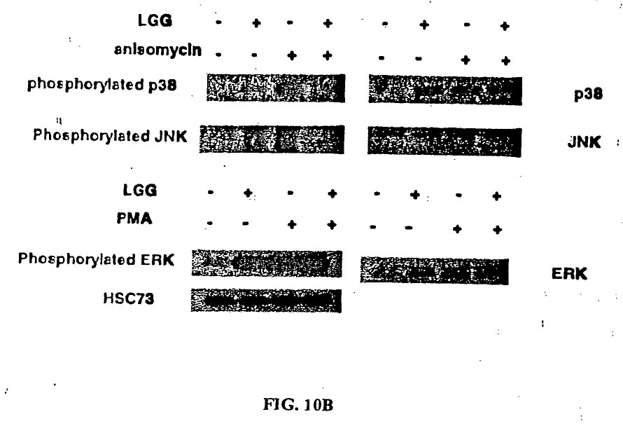

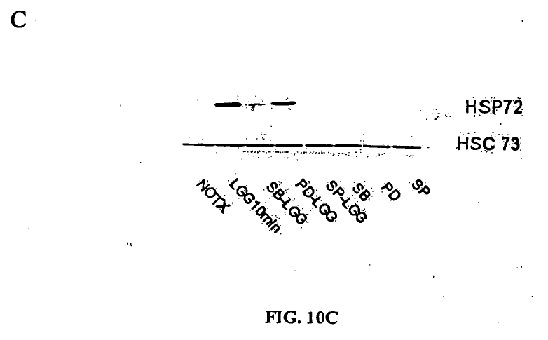

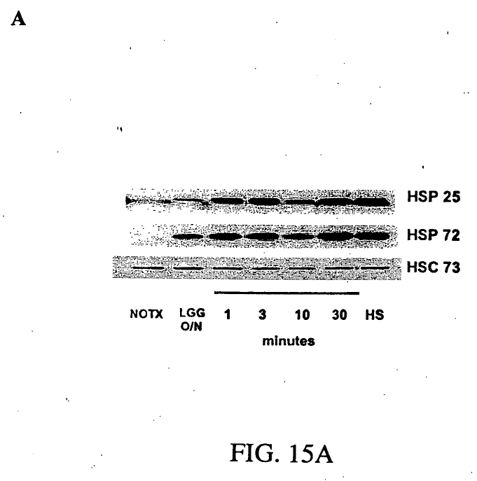

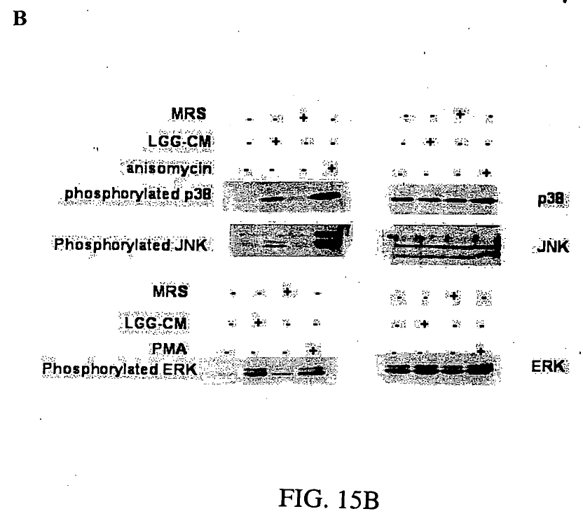

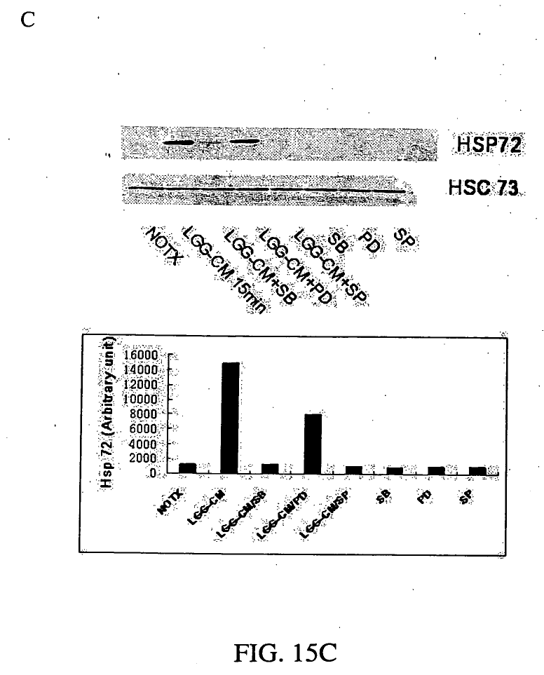

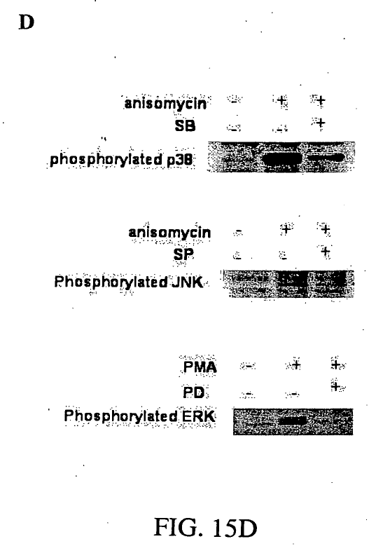

It was also determined whether induction of Hsps could be initiated after a transient exposure to LGG-CM. In other words, if the LGG-CM were washed off early in the course of treatment, the question was whether this transient exposure would be sufficient to cause induction of Hsps, or would Hsp induction require prolonged exposure to the LGG-CM. Cells were exposed to LGG-CM for short periods of time, the LGG-CM was washed off, then cells were harvested as usual and analyzed for Hsp production (FIGS. 10A and 15A). Even exposure times of a few minutes were sufficient to induce a robust response of Hsp induction, indicating that the time required to initiate the signal for the induction of heat shock proteins in epithelial cells is very short.

### Example 3

MAP Kinase Assays

To assess the participation of a signal transduction pathway in the Hsp expression induced by LGG-CM, MAP kinase assays were performed. For these assays, cells were plated at a density of 7.5×10⁵ cells per 100 mm dish. Cells were treated with LGG-CM and this was removed after 15 minutes and replaced with fresh RPMI medium. Cells were then harvested immediately after treatment with LGG-CM for Western blot analyses (MAP kinase phosphorylation). PVDF membranes were blocked in 3% weight/volume bovine serum albumin in TBS-Tween for one hour at room temperature. Primary antibodies were added to TBS-Tween and incubated overnight at 4° C. with antibodies specific for p38 MAP kinase (MAPK) #9212, Cell Signaling, Beverly, Mass.), phospho-p38 MAPK (#9211S, Cell Signaling), p44/42 MAPK (#9102, Cell Signaling), phospho-p44/42 ERK MAPK (#9101S), SAPK/JNK (#9252, Cell Signaling), and phospho-SAPK/JNK (#9251S, Cell Signaling). The phosphorylated form of the kinase indicates the activated form. As positive controls, 37.7 μM anisomycin (Alexis, San Diego, Calif.) was used for p38 and SAPK/JNK activation, and 100 nM phorbol 12-myristate 13-acetate (PMA [Sigma, St. Louis, Mo.]) was used for ERK1/2 activation.

Experiments with MAP kinase inhibitors confirmed the results obtained with the MAP kinase assays. In the MAP kinase inhibitor studies, YAMC cells were exposed to one of several known MAP kinase inhibitors, i.e., the p38 inhibitor SB203580 (20 μM; Alexis Biochemicals, Carlsbad, Calif.), the JNK inhibitor SP600125 (20 μM; Alexis Biochemicals), or the ERK inhibitor PD98059 (50 μM; Alexis Biochemicals), for two hours prior to addition of LGG-CM. Following addition of LGG-CM, cells were incubated for 15 minutes. Media was then replaced with fresh RPMI and YAMC cells were harvested four hours later for Western blot analyses of Hsps or immediately after LG-CM. This time point was chosen as this is the earliest point at which Hsp induction due to LGG-CM was typically seen. In all experiments, heat shock controls were heat-shocked at 42° C. for 23 minutes and then left at 37° C. for 2 hours before harvest.

Given the rapidity of the response to LGG-CM, the data are consistent with the involvement of signal transduction pathways in elaborating the response in epithelial cells. To investigate this possibility, cells were treated with LGG-CM for 15 minutes and then kinase assays were performed.

Many protein kinases are known to be activated by stresses such as LPS, TNFα, heat, ultraviolet radiation, chemicals and osmotic shock, and several of these kinases belong to the MAP kinase family (Keyse, Stress Response: methods and protocols, Totowa: Humana Press, 2000). Accordingly, the effects on this group of kinases were chosen as a readout for signal transduction activation. Even after short exposure times, differences in kinase activation between treated and untreated cells were apparent (FIGS. 10B and 15B). Pretreatment of cells with LGG-CM alone activates all three MAP kinases investigated. Although there is a baseline level of activated ERK1/2 in YAMC cells, the activation of ERK1/2 by LGG-CM was almost as robust as activation by the phorbol ester PMA, whereas LGG-CM treatment resulted in a clear, but less dramatic, activation of p38 and JNK than was seen with anisomycin, a known potent stimulator of p38 and SAP/JNK activation. Inhibitors of all three MAP kinases investigated were used to determine if activation of a MAP kinase pathway was required for Hsp induction by LGG-CM. Exposure of YAMC cells to inhibitors of p38 and JNK prior to LGG-CM treatment resulted in blockade of Hsp72 expression, thus confirming a role for MAP kinase signaling pathways in the induction of Hsps by LGG-Cm in epithelial cells (FIGS. 10C and 15C). Densitometry of these immunoblots indicates that PD98059 has a more modest effect on inhibiting Hsp72 expression than do the p38 and JNK inhibitors, suggesting that ERK plays a lesser role. The specificity of the MAP kinase inhibitors for their respective kinases was verified and is shown in FIG. 15D.

The cytoprotection conferred by Hsp72 (Hsp70) under conditions of stress has been reported to act, in part, through inhibition of p38 and JNK, which confers resistance to stress-induced cell death (Gabai et al., J Biol Chem 272: 18033-18037, 1997; Mosser et al., Mol Cell Biol 17: 5317-5327, 1997). The data disclosed herein, however, establish that inhibition of p38 and JNK activation seen after LGG-CM treatment could not possibly be due to Hsp72 (Hsp70) because the effect occurred within such a short time period, and appearance of Hsps after LGG-CM treatment takes several hours. Activation of JNK has been shown to play an important role in mediating cell death under conditions of chemical and physical stress, and blockade of JNK confers resistance to cell death induced by various forms of stress (Zanke et al., Curr Biol 6: 606-613, 1996). Similar observations have been made for the kinase p38 (Gabai et al., 1997), and studies indicate that both p38 and JNK act through a common pathway that is distinct from ERK1/2 (Liu et al., Free Radic Biol Med 21: 771-781, 1996). Hence, it is expected that the soluble factors in LGG-CM possess their own cytoprotective properties, which act through yet other mechanisms, in addition to their abilities to induce cytoprotective Hsps.

In contrast to a report by Yan et al., 2002, Lactobacillus GG does produce at least one bioactive factor that is recoverable from conditioned media. Growth studies revealed that it takes at least 8 hours for the bacteria to produce these bioactive factor(s) when grown in MRS medium and the organism does not produce these bioactive factor(s) when grown in tissue culture media. Also, E. coli LPS has been reported as inducing Hsp25 through MAP kinase activation in YAMC cells (Kojima et al., 2004), but significant differences between that study and the current disclosure are that, first, Lactobacillus GG is a Gram-positive organism and, therefore, contains no LPS, and, second, E. coli LPS does not induce Hsp72, whereas Hsp72 (and Hsp25) is induced by LGG-CM through a MAP kinase-dependent pathway in YAMC cells.

### Example 4

RNA Isolation and Reverse Transcription

Cells were washed twice in ice-cold HBS and harvested as described above, then 1.0 ml TRIzol® (Invitrogen, Carlsbad, Calif.) was added as per the manufacturer's instructions and 200 μL of chloroform (Fisher, Fair Lawn, N.J.) were added per 1 ml of TRIzol used for homogenization and the material was centrifuged at 14,000×g for 15 minutes at 4° C. The aqueous phase was removed and RNA was precipitated using isopropanol, and then washed twice with 75% ethanol. The RNA pellet was dried, dissolved in RNase-free water and then further purified using an RNeasy spin column (QIAGEN, Valencia, Calif.) as per the manufacturer's instructions. Sample integrity was analyzed on 1% agarose gels and by absorbance at 280 nm and 260 nm. The cDNA was synthesized using SuperScript II RT (Invitrogen, Carlsbad, Calif.). The reverse transcriptase reaction was performed using 3 μg of total RNA in a total volume of 20 μl containing the following: 1× first-strand buffer, 250 ng random hexanucleotide primer, 3 μg RNA, 500 μM dNTPs, 10 mM DTT, 40 units of Rnase out Ribonuclease inhibitor, and 200 units of SuperScriptIIRT. The reaction mixture was incubated at 25° C. for 10 minutes, then at 42° C. for 50 minutes, and the reverse transcriptase was then inactivated by heating at 70° C. for 15 minutes. The cDNA was used as a template for amplification by PCR. The cDNA samples were diluted to 1:5 and stored at −20° C. for further study. RNA was precipitated using isopropanol and then washed twice with 75% ethanol/DEPC-treated water. Sample integrity was analyzed on 1% agarose gels and by analysis of the UV wavelength absorbance at 280 nm and 260 nm; this ratio was then used to verify RNA purity, as known in the art.

### Example 5

Real-Time PCR

The time course of Hsp expression was determined using real-time PCR. Primers for the mouse Hsp25 and Hsp72 coding regions were designed using sequences downloaded from Genbank. The primers were designed by using Primer Express Software (Applied Biosystems, Foster City, Calif.). The sense and antisense primers for mouse Hsp25 are: 5′-CCA TGT TCG TCC TGC CTT TC-3′ (SEQ ID NO:1) and 5′-GAG GGC TGC TTC TGA CCT TCT-3′ (SEQ ID NO:2); for mouse Hsp72: 5′-GGC TGA TCG GAC GGA AGT T-3′ (SEQ ID NO:3) and 5′-GGA ACG GCC AGT GCT TCA T-3′ (SEQ ID NO:4); for mouse GAPDH: 5′-GGC AAA TTC AAC GGC ACA GT-3′ (SEQ ID NO:5) and 5′-AGA TGG TGA TGG GCT TCC C-3′ (SEQ ID NO:6). Real-time PCR was performed in triplicate in an iCycler (Bio-Rad, Hercules, Calif.) with iQSYBR Green PCR supermix (Bio-Rad, Hercules, Calif.). Direct detection of PCR product was monitored by measuring the increase in fluorescence caused by the binding of SYBR Green dye to double-stranded (ds) DNA. A final volume of 25 μl contained 1× SYBR Green PCR supermix and primers at a final concentration of 300 nM. Three microliters of diluted (1:5) cDNA were added to 23 μl of the PCR master mixture. The following quantification cycling protocol was used: 4 minutes at 95° C. to activate Taq DNA polymerase, followed by 45 cycles of denaturation at 95° C. for 15 seconds and annealing-extension at 60° C. for 15 seconds. The threshold cycle parameter (Ct) was defined as the fractional cycle number at which the fluorescence crossed a fixed threshold above the baseline. ΔCt value was determined by subtracting the average GAPDH Ct value from the average Hsp 25 or Hsp72 Ct value. The ΔΔCt calculation was used for the relative quantitation of target without running standard curves on the sample plate. This involved the subtraction of an arbitrary constant, so the standard deviation of ΔΔCt was the same as the standard deviation of the ΔCt value. The fold change in YAMC RNA (target gene) relative to the GAPDH endogenous control was determined using the following equation: Fold Change=2^{-ΔΔCt}.

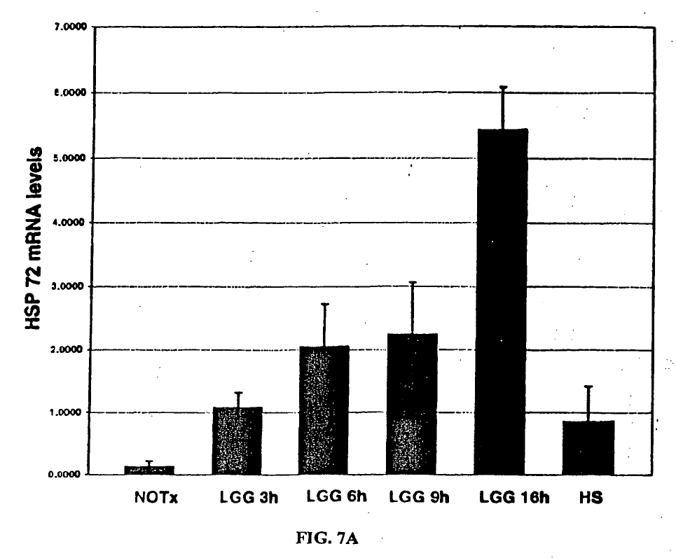

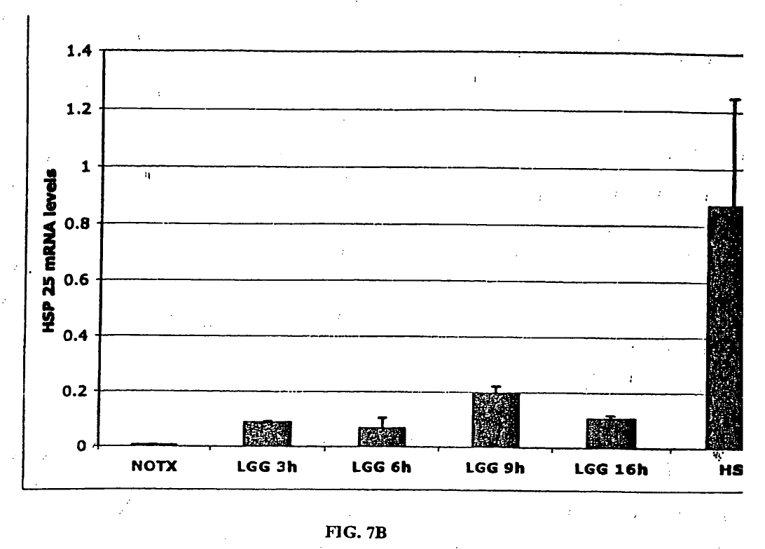

Using real-time PCR, it was found that mRNA levels for both Hsp25 and Hsp72 increased after LGG-CM treatment, indicating that the induction of these two Hsps by LGG-CM was transcriptional in nature (FIG. 7).

### Example 6

Electrophoretic Mobility Shift Assays

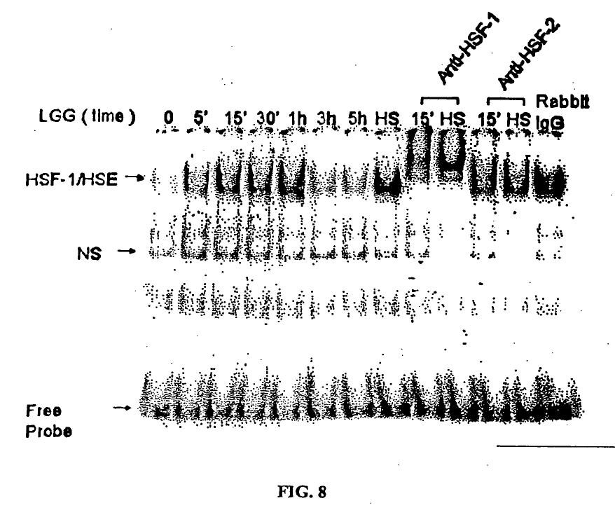

To further investigate the nature of Hsp induction by LGG-CM, electrophoretic mobility shift assays (EMSAs) were performed (FIG. 8). Cells were either treated with LGG-conditioned media (LLG-CM), or heat-shocked as described above. Whole cell extracts were prepared in lysis buffer (25% vol/vol glycerol, 420 mM NaCl, 1.5 mM MgCl2, 0.2 mM EDTA, 0.5 mM DTT, 20 mM HEPES, pH 7.4 with the Complete Protease Inhibitor Cocktail) by freezing once in a dry ice/alcohol bath, thawing on ice, shearing gently with a pipette tip, and centrifugation at 50,000×g for 5 minutes at 4° C. (See Mosser et al. (1988), incorporated herein by reference). Ten micrograms of whole cell extract were mixed with γ-³²P-ATP-labeled HSE oligonucleotide (containing four tandem inverted repeats of the heat shock element (nGAAn): 5′-CTAGAAGCTTCTAGAAGCTTCTAG-3′; SEQ ID NO:7) and 0.5 μg poly (dI-dC) plus 20 units of T4 polynucleotide kinase (New England Biolabs, Beverly, Mass.) in 1× binding reaction buffer (final concentrations of 20 mM Tris, pH 7.4, 100 mM NaCl, 1 mM EDTA, 10% vol/vol glycerol). The binding reaction was allowed to incubate for 60 minutes at 37° C. and labeled oligonucleotide was separated from free probe using G50 spin columns (Amersham Biosciences, Piscataway, N.J.) following the manufacturer's instructions. Annealing of labeled oligonucleotide and unlabeled oligonucleotide strands was performed at 95° C. for 5 minutes, then allowed to cool slowly overnight. Samples were then analyzed on a 4% non-denaturing polyacrylamide gel run in 0.5× TBE buffer. Gels were dried and autoradiographed to detect DNA-protein complexes. For supershift experiments, YAMC cells were incubated with LGG-CM and then 1 μg of either rat monoclonal anti-HSF-1 antibody (SPA 950, Stressgen, Victoria, BC, Canada), 1 μg rat monoclonal anti-HSF-2 (SPA 960, Stressgen), or 1 μl of rabbit pre-immune serum was pre-incubated with cell extracts at 25° C. for 30 minutes prior to the HSE-binding reaction. After this preincubation, the binding reaction and analysis were performed using standard methodologies known in the art or described herein.

Results demonstrated that binding of HSF-1 in response to LGG-CM occurs within the first hour of exposure to LGG-CM, indicating that the induction is at least partly transcriptional in nature (FIG. 7). Supershift analysis with antibodies to HSF-1 and HSF-2 showed that HSF-1 is the principal transcription factor involved (FIG. 8).

Transient exposure to LGG-CM results in increased Hsp expression by a mechanism that is, at least in part, transcriptional in nature and involves the transcription factor Heat Shock Factor-1 (HSF-1), since even short LGG-CM exposure times of only 5 minutes results in activation of HSF-1. Also, the effects of LGG-CM on Hsp25 mRNA appear quite modest compared to the large fold induction of Hsp25 protein. The apparent discrepancy between amount of mRNA induction and level of protein induction seen for Hsp25 suggests that there may be post-transcriptional mechanisms of regulation involved, as has been described for other genes such as Cox-2 (Dixon et al., 2000).

### Example 7

Microarray Analyses

Hsps are the most highly upregulated genes in response to LGG-CM exposure. After having established that LGG-CM treatment induced a robust heat shock protein induction and that the mechanism behind Hsp induction by LGG-CM in epithelial cells was at least largely transcriptional in nature, the magnitude of the upregulation of Hsps compared to other epithelial cell genes was determined by DNA microarray analysis. LGG-CM-treated and MRS-treated (mock-treated) cells were compared using two different microarray chips, one containing 19,000 murine gene probes and the other containing 12,000 murine gene probes.

RNA was prepared as described above and then subjected to one additional purification step using the RNeasy Mini Kit (QIAGEN, Valencia, Calif.) as per the manufacturer's instructions. RNA integrity was checked by fractionation on a 1% agarose. gel. Only RNA with a 280 nm/260 nm ratio between 1.8 and 2.0 was used.

An Affymetrix microarray chip 430A containing 19,000 murine genes was run in duplicate; the U74Av2 chip containing 12,000 murine genes but using different probe sets was used to confirm the results obtained with chip 430A. Data were analyzed using Affymetrix Genechip Operating Software (GCOS, Version 1.0). In each case, LGG treatment was compared to mock-treatment controls. Results were expressed as fold change of treated cells as compared to controls, as calculated using GENESPRING software (Version 4.2.1, Silicon Genetics, Mountain View, Calif.). Statistical analysis was performed using D chip software. See Tusher et al. (2001) and Li et al. (2001). Differentially expressed genes were selected based on the following thresholds: relative difference greater than 1.5 fold, absolute difference greater than 100 signal intensity units and statistical difference p<0.05. Data from the Affymetrix 430A microarray chips has been deposited in the Gene Expression Omnibus data repository accessible via the internet (see series entry, GSE 1940).

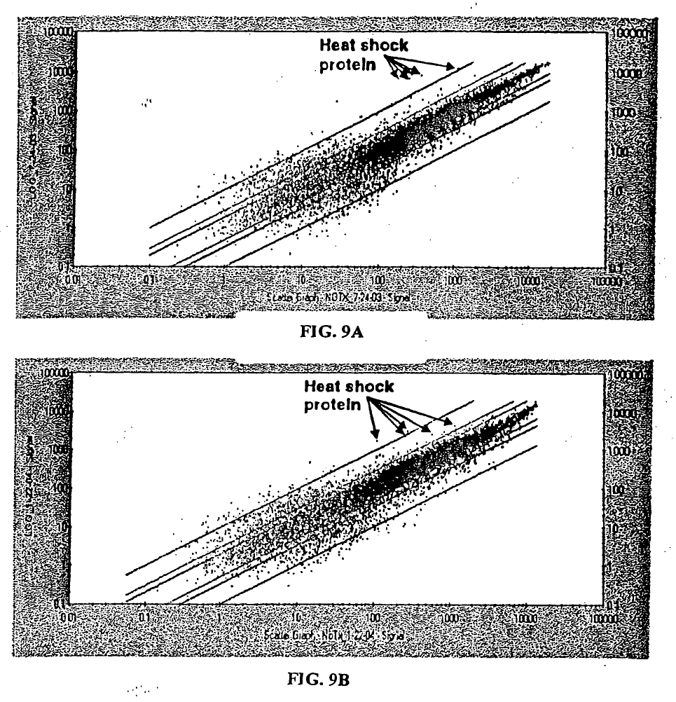

It can be seen from the scatter plots that the most dramatically upregulated genes in response to LGG-CM treatment were the heat shock protein genes (FIG. 9 and Table). To confirm these findings, an additional gene chip containing 12,000 murine genes and using different probe sets was used and, again, Hsps were found to be the most upregulated genes in response to LGG-CM treatment. The top ten upregulated genes are presented in Table 6, below.

Twenty-four genes that exhibited a greater than 2-fold change between LGG-CM-treated cells and controls on both chip 430A and chip U74Av2 are listed in Table 1. All of the sequences corresponding to the GenBank Accession numbers listed in Table 1 and listed in the preceding paragraph are incorporated herein by reference.

TABLE 1

| | | | GenBank | Fold |
|---|---|---|---|---|
| Chip | Probe Set | Gene | Accession | Change |
| 430A | 1416041_at | serum/glucocorticoid regulated kinase | NM_011361 | 4.15 |
| U74Av2 | 97890_at | serum/glucocorticoid regulated kinase | AW046181 | 3.48 |
| 430A | 1416855_at | growth arrest specific 1 | NM_008086 | −2.69 |
| 430A | 1448494_at | growth arrest specific 1 | BB550400 | −2.33 |
| U74Av2 | 94813_at | growth arrest specific 1 | X65128 | −3.06 |
| 430A | 1417516_at | DNA-damage inducible transcript 3 | NM_007837 | 4.04 |
| U74Av2 | 101429_at | DNA-damage inducible transcript 3 | X67083 | 10.55 |
| 430A | 1453851_a_at | growth arrest and DNA-damage- | AK007410 | 2.45 |
| | | inducible 45 gamma | | |
| U74Av2 | 101979_at | growth arrest and DNA-damage- | AF055638 | 2.73 |
| | | inducible 45 gamma | | |
| 430A | 1448830_at | dual specificity phosphatase 1 | NM_013642 | 3.34 |
| U74Av2 | 104598_at | dual specificity phosphatase 1 | X61940 | 3.01 |
| 430A | 1418930_at | Chemokine (C-X-C motif) ligand 10 | NM_021274 | 2.47 |
| U74Av2 | 93858_at | Chemokine (C-X-C motif) ligand 10 | M33266 | 4.72 |
| 430A | 1449363_at | activating transcription factor 3 | BC019946 | 3.76 |
| U74Av2 | 104155_f_at | activating transcription factor 3 | U19118 | 5.76 |
| 430A | 1419149_at | serine (or cysteine) proteinase | NM_008871 | 3.15 |
| | | inhibitor, clade E, member 1 | | |
| U74Av2 | 94147_at | serine (or cysteine) proteinase | M33960 | 2.43 |
| | | inhibitor, clade E, member 1 | | |
| 430A | 1419291_x_at | growth arrest specific 5 | NM_013525 | 2.44 |
| U74Av2 | 98531_g_at | growth arrest specific 5 | AI849615 | 2.61 |
| 430A | 1449519_at | growth arrest and DNA-damage- | NM_007836 | 3.28 |
| | | inducible 45 alpha | | |
| U74Av2 | 102292_at | growth arrest and DNA-damage- | U00937 | 4.09 |
| | | inducible 45 alpha | | |
| 430A | 1419665_a_at | nuclear protein 1 | NM_019738 | 3.36 |
| 430A | 1419666_x_at | nuclear protein 1 | NM_019738 | 2.79 |
| U74Av2 | 160108_at | nuclear protein 1 | AI852641 | 5.8 |
| 430A | 1422557_s_at | metallothionein 1 | NM_013602 | −2.15 |
| U74Av2 | 93573_at | metallothionein 1 | V00835 | 2.53 |
| 430A | 1422990_at | met proto-oncogene | NM_008591 | −3.43 |
| U74Av2 | 100309_at | met proto-oncogene | Y00671 | −2.68 |
| 430A | 1423062_at | insulin-like growth factor binding | AV175389 | −2.35 |
| | | protein 3 | | |
| U74Av2 | 95082_at | insulin-like growth factor binding | AI842277 | −3.74 |
| | | protein 3 | | |
| 430A | 1423100_at | FBJ osteosarcoma oncogene | AV026617 | 2.96 |
| U74Av2 | 160901_at | FBJ osteosarcoma oncogene | V00727 | 4.54 |
| 430A | 1451313_a_at | RIKEN cDNA 1110067D22 gene | BC019131 | 2.32 |
| U74Av2 | 160704_at | RIKEN cDNA 1110067D22 gene | AW121603 | 2.17 |
| 430A | 1426559_at | cDNA sequence BC021875 | BG065326 | 2.59 |
| U74Av2 | 104106_at | cDNA sequence BC021875 | AI837830 | −5.64 |
| 430A | 1427585_at | Mus musculus DNA cytosine | AF071754 | 25.2 |
| | | methyltransferase mRNA | | |
| U74Av2 | 95396_at | Mus musculus DNA cytosine | AF071754 | 5.86 |
| | | methyltransferase mRNA | | |
| 430A | 1428529_at | RIKEN cDNA 2810026P18 gene | AK012825 | 2.18 |
| U74Av2 | 104089_at | RIKEN cDNA 2810026P18 gene | AW045664 | 3.01 |
| 430A | 1430271_x_at | RIKEN cDNA 4930553M18 gene | AA672926 | 2.07 |
| U74Av2 | 104640_f_at | RIKEN cDNA 4930553M18 gene | AI464596 | 2.1 |
| 430A | 1436549_a_at | heterogeneous nuclear | BE685966 | 2.17 |
| | | ribonucleoprotein A1 | | |
| U74Av2 | 92724_at | heterogeneous nuclear | AI183202 | 2.08 |
| | | ribonucleoprotein A1 | | |
| 430A | 1436791_at | wingless-related MMTV integration | BB067079 | −2.61 |
| | | site 5A | | |
| U74Av2 | 99390_at | wingless-related MMTV integration | M89798 | −2.16 |
| | | site 5A | | |
| 430A | 1455904_at | growth arrest specific 5 | BI650268 | 2.66 |
| U74Av2 | 98531_g_at | growth arrest specific 5 | AI849615 | 2.61 |
| 430A | 1449773_s_at | growth arrest and DNA-damage- | AI323528 | 2.41 |
| | | inducible 45 beta | | |
| U74Av2 | 161666_f_at | growth arrest and DNA-damage- | AV138783 | 2.52 |
| | | inducible 45 beta | | |

Four common gene ontology groups between chip 430A and chip U74Av2 are shown in Tables 2 to 5. Only genes that exhibited a greater than 2-fold change between LGG-CM-treated cells and controls are shown. All sequences corresponding to Genbank numbers listed in Tables 2 to 5 are incorporated herein by reference.

TABLE 2
Regulation of Cell Cycle Genes.
For the 430A chip, 14 gene ontology “regulation of cell cycle” genes
were found in a 125-group (all: 212/13281, p-value: 0.000000). For the U74Av2 chip,
10 gene ontology “regulation of cell cycle” genes were found in a 96-group
(all: 146/6741, p-value: 0.000038).

| | | | GenBank | Fold |
|---|---|---|---|---|
| Chip | Probe Set | Gene | Accession | Change |
| 430A | 1416855_at | growth arrest specific 1 | NM_008086 | −2.69 |
| | 1448494_at | growth arrest specific 1 | BB550400 | −2.33 |
| | 1417516_at | DNA-damage inducible transcript 3 | NM_007837 | 4.04 |
| | 1418936_at | v-maf musculoaponeurotic | BC022952 | 2.27 |
| | | fibrosarcoma oncogene family, | | |
| | | protein F (avian) | | |
| | 1419291_x_at | growth arrest specific 5 | NM_013525 | 2.44 |
| | 1449519_at | growth arrest and DNA-damage- | NM_007836 | 3.28 |
| | | inducible 45 alpha | | |
| | 1450016_at | cyclin G1 | NM_009831 | −2.65 |
| | 1450017_at | cyclin G1 | BG065754 | −2.35 |
| | 1421679_a_at | cyclin-dependent kinase inhibitor 1A | NM_007669 | −2.15 |
| | | (P21) | | |
| | 1450533_a_at | pleiomorphic adenoma gene-like 1 | NM_009538 | −3.05 |
| | 1422990_at | met proto-oncogene | NM_008591 | −3.43 |
| | 1423100_at | FBI osteosarcoma oncogene | AV026617 | 2.96 |
| | 1426208_x_at | pleiomorphic adenoma gene-like 1 | AF147785 | −2.22 |
| | 1455904_at | growth arrest specific 5 | BI650268 | 2.66 |
| U74Av2 | 94813_at | growth arrest specific 1 | X65128 | −3.06 |
| | 98531_g_at | growth arrest specific 5 | AI849615 | 2.61 |
| | 103048_at | neuroblastoma myc-related oncogene 1 | M12731 | −2.09 |
| | 94338_g_at | growth arrest specific 2 | M21828 | 3.23 |
| | 101429_at | DNA-damage inducible transcript 3 | X67083 | 10.55 |
| | 102292_at | growth arrest and DNA-damage- | U00937 | 4.09 |
| | | inducible 45 alpha | | |
| | 92502_at | zinc finger protein regulator of | X95504 | −2.56 |
| | | apoptosis and cell cycle arrest | | |
| | 95348_at | chemokine (C-X-C motif) ligand 1 | J04596 | −2.61 |
| | 100309_at | met proto-oncogene | Y00671 | −2.68 |
| | 160901_at | FBJ osteosarcoma oncogene | V00727 | 4.54 |

TABLE 3
Cell Cycle Genes.
For the 430A chip, 18 gene ontology “cell cycle” genes were found
in a 125-group (all: 465/13281, p-value: 0.000000). For the U74Av2 chip,
14 gene ontology “cell cycle” genes were found in a 96-group
(all: 326/6741, p-value: 0.000188).

| | | | GenBank | Fold |
|---|---|---|---|---|
| Chip | Probe Set | Gene | Accession | Change |
| 430A | 1416120_at | ribonucleotide reductase M2 | BF119714 | −2.31 |
| | 1448458_at | topoisomerase (DNA) II beta | BB166592 | −2.4 |
| | 1416855_at | growth arrest specific 1 | NM_008086 | −2.69 |
| | 1448494_at | growth arrest specific 1 | BB550400 | −2.33 |
| | 1417516_at | DNA-damage inducible transcript 3 | NM_007837 | 4.04 |
| | 1448830_at | dual specificity phosphatase 1 | NM_013642 | 3.34 |
| | 1418936_at | v-maf musculoaponeurotic | BC022952 | 2.27 |
| | | fibrosarcoma oncogene family, | | |
| | | protein F (avian) | | |
| | 1419291_x_at | growth arrest specific 5 | NM_013525 | 2.44 |
| | 1449519_at | growth arrest and DNA-damage- | NM_007836 | 3.28 |
| | | inducible 45 alpha | | |
| | 1450016_at | cyclin G1 | NM_009831 | −2.65 |
| | 1450017_at | cyclin G1 | BG065754 | −2.35 |
| | 1421679_a_at | cyclin-dependent kinase inhibitor 1A | NM_007669 | −2.15 |
| | | (P21) | | |
| | 1450533_a_at | pleiomorphic adenoma gene-like 1 | NM_009538 | −3.05 |
| | 1422990_at | met proto-oncogene | NM_008591 | −3.43 |
| | 1423100_at | FBJ osteosarcoma oncogene | AV026617 | 2.96 |
| | 1426208_x_at | pleiomorphic adenoma gene-like 1 | AF147785 | −2.22 |
| | 1434496_at | cytokine inducible kinase | BM947855 | 2.6 |
| | 1455904_at | growth arrest specific 5 | BI650268 | 2.66 |
| U74Av2 | 94813_at | growth arrest specific 1 | X65128 | −3.06 |
| | 98531_g_at | growth arrest specific 5 | AI849615 | 2.61 |
| | 103048_at | neuroblastoma myc-related oncogene 1 | M12731 | −2.09 |
| | 104598_at | dual specificity phosphatase 1 | X61940 | 3.01 |
| | 94338_g_at | growth arrest specific 2 | M21828 | 3.23 |
| | 101429_at | DNA-damage inducible transcript 3 | X67083 | 10.55 |
| | 101930_at | nuclear factor I/X | Y07688 | −2.4 |
| | 102292_at | growth arrest and DNA-damage- | U00937 | 4.09 |
| | | inducible 45 alpha | | |
| | 92502_at | zinc finger protein regulator of | X95504 | −2.56 |
| | | apoptosis and cell cycle arrest | | |
| | 95348_at | chemokine (C-X-C motif) ligand 1 | J04596 | −2.61 |
| | 100309_at | met proto-oncogene | Y00671 | −2.68 |
| | 101180_at | ataxia telangiectasia mutated | U43678 | −2.49 |
| | | homolog (human) | | |
| | 160859_s_at | nuclear factor I/B | Y07685 | −2.27 |
| | 160901_at | FBJ osteosarcoma oncogene | V00727 | 4.54 |

TABLE 4
Cell Cycle Arrest Genes.
For the 430A chip, 7 gene ontology “cell cycle arrest” genes were found
in a 125-group (all: 26/13281, p-value: 0.000000). For the U74Av2 chip,
5 gene ontology “cell cycle arrest” genes were found in a 96-group
(all: 22/6741, p-value: 0.000011).

| | | | GenBank | Fold |
|---|---|---|---|---|
| Chip | Probe Set | Gene | Accession | Change |
| 430A | 1416855_at | growth arrest specific 1 | NM_008086 | −2.69 |
| | 1448494_at | growth arrest specific 1 | BB550400 | −2.33 |
| | 1417516_at | DNA-damage inducible transcript 3 | NM_007837 | 4.04 |
| | 1419291_x_at | growth arrest specific 5 | MM_013525 | 2.44 |
| | 1449519_at | growth arrest and DNA-damage- | NM_007836 | 3.28 |
| | | inducible 45 alpha | | |
| | 1421679_a_at | cyclin-dependent kinase inhibitor 1A | NM_007669 | −2.15 |
| | | (P21) | | |
| | 1455904_at | growth arrest specific 5 | BI650268 | 2.66 |
| U74Av2 | 94813_at | growth arrest specific 1 | X65128 | −3.06 |
| | 98531_g_at | growth arrest specific 5 | AI849615 | 2.61 |
| | 94338_g_at | growth arrest specific 2 | M21828 | 3.23 |
| | 101429_at | DNA-damage inducible transcript 3 | X67083 | 10.55 |
| | 102292_at | growth arrest and DNA-damage- | U00937 | 4.09 |
| | | inducible 45 alpha | | |

TABLE 5
Ribosomal Protein L7Ae/L30e/Gadd45 Genes.
For the 430A chip, 3 gene ontology “ribosomal protein L7Ae/L30e/S12e/Gadd45”
genes were found in a 118-group (all: 14/13714, p-value: 0.000211). For the U74Av2 chip,
3 gene ontology “ribosomal protein L7Ae/L30e/S12e/Gadd45” genes were found in
a 99-group (all: 7/7501, p-value: 0.000075).

| | | | GenBank | Fold |
|---|---|---|---|---|
| Chip | Probe Set | Gene | Accession | Change |
| 430A | 1453851_a_at | growth arrest and DNA-damage- | AK007410 | 2.45 |
| | | inducible 45 gamma | | |
| | 1449519_at | growth arrest and DNA-damage- | NM_007836 | 3.28 |
| | | inducible 45 alpha | | |
| | 1449773_s_at | growth arrest and DNA-damage- | AI323528 | 2.41 |
| | | inducible 45 beta | | |
| U74Av2 | 101979_at | growth arrest and DNA-damage- | AF055638 | 2.73 |
| | | inducible 45 gamma | | |
| | 102292_at | growth arrest and DNA-damage- | U00937 | 4.09 |
| | | inducible 45 alpha | | |
| | 161666_f_at | growth arrest and DNA-damage- | AV138783 | 2.52 |
| | | inducible 45 beta | | |

For these microarray studies, the statistical analyses were performed using “D chip” and “Sam” software as described in Tusher et al. (2001) and Li et al. (2001), both incorporated herein by reference.

TABLE 6

| | Relative | | |
|---|---|---|---|
| | expression | | |
| | change | | |
| | (fold | | |
| Genbank | change, | | |
| Accession | LGG-CM v. | | |
| Number | nothing) | P Value | Gene description |
| AW763765 | 68.59 | 0.00002 | Mouse inducible heat |
| | | | shock protein, 70 kDa |
| | | | 3 (i.e., Hsp72, alter- |
| | | | natively referenced |
| | | | as Hsp70) |
| M12573.1 | 36.76 | 0.00002 | Mouse inducible heat |
| | | | shock protein, 70 kDa |
| | | | 1 (Hsp68, the human |
| | | | homolog of mouse |
| | | | Hsp72/Hsp70) |
| U03561.1 | 22.63 | 0.00002 | Mouse heat shock protein |
| | | | Hsp27 internal deletion |
| | | | variant b |
| AF071754.1 | 13.93 | 0.000492 | Mouse DNA cytosine |
| | | | methyltransferase |
| AK013777.1 | 13.00 | 0.00003 | Protein tyrosine |
| | | | phosphatase, non- |
| | | | receptor type 21 |
| BC025911.1 | 12.13 | 0.000241 | Mouse sorting nexin 6 |
| NM_013560.1 | 11.31 | 0.00002 | Mouse heat shock protein, |
| | | | 25 kDa (Hsp25, the human |
| | | | homolog of mouse Hsp25). |
| L07264.1 | 11.31 | 0.00002 | Mouse heparin-binding |
| | | | EGF-like growth factor |
| | | | precursor |
| BH320427 | 11.31 | 0.001832 | RIKEN cDNA 5430423014 |
| | | | gene (EST, gene |
| | | | function unknown) |
| L25109.1 | 10.56 | 0.000147 | Mouse lissencephaly gene |
| | | | (LIS1) partial cds. |

### Example 8

⁵¹Chromium Release Assays

LGG-CM also protects epithelial cells against oxidant damage. Given that LGG-CM upregulates inducible Hsps, functional assays were undertaken to determine whether heat shock induction contributed to protection against oxidant damage. Normally produced when hypochlorous acid released from innate immune cells reacts with ammonia, the oxidant monochloramine affects epithelial cells by causing cytoskeletal disruption, impaired membrane transport, loss of tight barrier function, and eventual cell death (Grisham et al., 1990; Musch et al., 1996; Musch et al., 1999). Studies have shown that inducible Hsps provide cytoprotection against the oxidant stress caused by monochloramine in gut epithelial cells (Musch et al., 1996; Musch et al., 1999).

YAMC cells were grown in 24-well plates and either left untreated (control), or treated with LGG-CM for one hour and then the media was replaced and the cells were left overnight at 37° C. in a 5% CO₂ incubator. Cells were then loaded with ⁵¹Cr (50 μCi/ml; Sigma Chemical Co.) for 60 minutes, washed, and incubated in media with 0.6 mM of the oxidant monochloramine to induce cell injury (Musch et al., 1996; Musch et al., 1999). After 60 minutes, media was harvested and the ⁵¹Cr remaining in the cells was extracted with 1N HNO₃ for 4 hours. ⁵¹Cr in the released and cellular fractions was counted by liquid scintillation spectroscopy. ⁵¹Cr released was calculated as the amount released divided by the sum of released plus cellular remainder. The data were compiled and analyzed using Instat software (Graphpad, San Diego, Calif.) and comparisons were made using the paired Student's T-test.

For silencing of LGG-induced Hsps, YAMC cells were plated and allowed to grow for 24 hours in complete medium. Twenty-five mer silencing oligonucleotides were designed for Hsp25 (corresponding to nucleotides 1266-1290 and 1503-1527 of mouse Hsp25, GenBank Accession Number L07577) or Hsp72 (nucleotides 1691-1715 of human Hsp72, GenBank Accession Number M1717) using Invitrogen RNAi designer software (Carlsbad, Calif.). For each well, sufficient oligonucleotide for a final concentration of 20 nM (in 500 μl final volume) was added to 100 μl of Opti-Mem medium (Invitrogen, Grand Island, N.Y.) and mixed with 0.4 μl of SilentFect Reagent (BioRad, Hercules, Calif.) in 100 μl Opti-Mem and allowed to complex for 20 minutes at room temperature. Medium was removed from the cells and the oligo/SilentFect mixture in Opti-Mem was added to the well and incubated for 60 minutes. At this time, 300 μl of complete medium was added and the cells were allowed to grow for either 48 hours (for Hsp72 siRNA) or were re-pulsed with siRNA after 24 hours (for Hsp25 siRNA) and allowed to grow for an additional 24 hours. Cells were treated with LGG-CM as described herein one day prior to chromium loading and NH₂Cl injury as described herein. When inhibitors of MAP kinases were used, cells were treated with PD98059 (50 μM), SB203580 (20 μM), or SP600125 (20 μM) for two hours prior to addition of LGG-CM.

After one hour, LGG-CM was added and cells were left in LGG-CM-containing medium for one hour. The cell culture medium containing inhibitors and LGG-CM was then replaced with fresh medium (i.e., no inhibitors or LGG-CM) and cells were returned to the incubator overnight before chromium loading and injury.

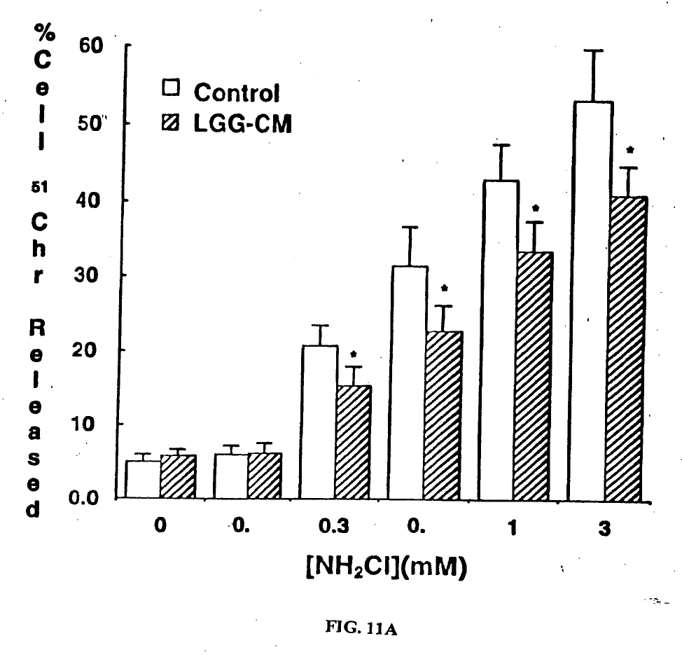

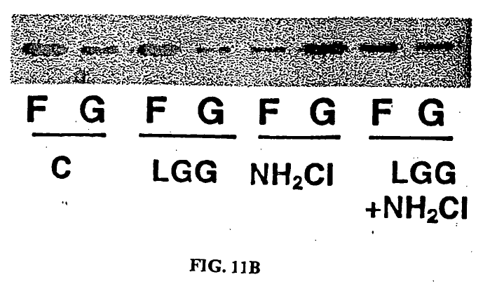

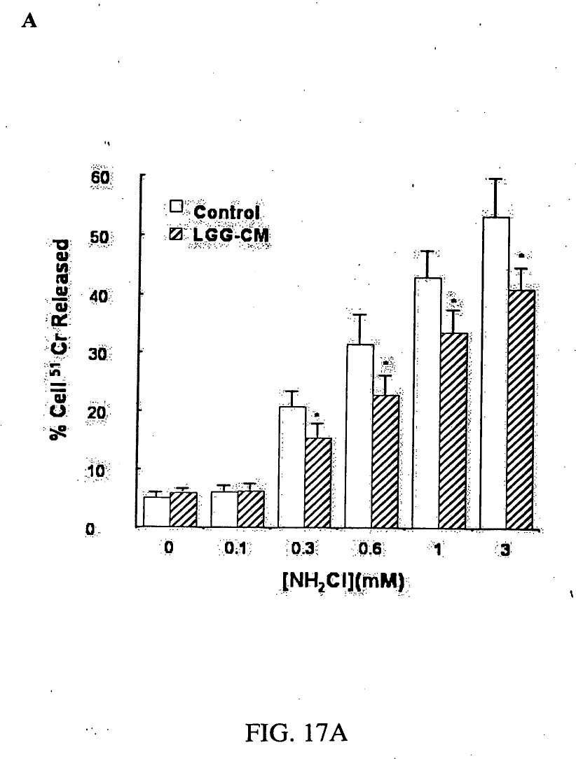

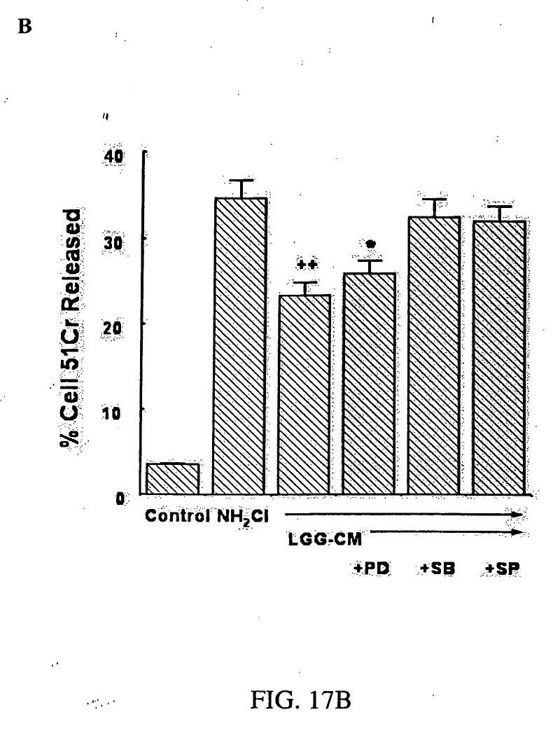

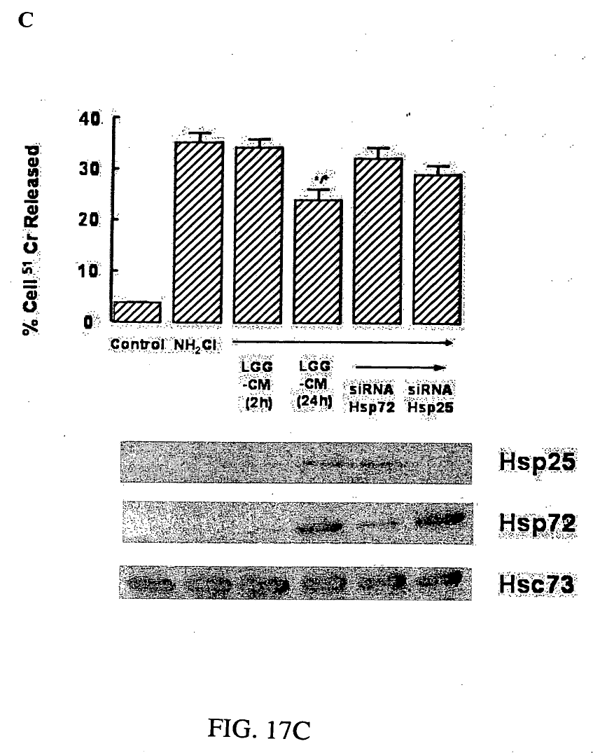

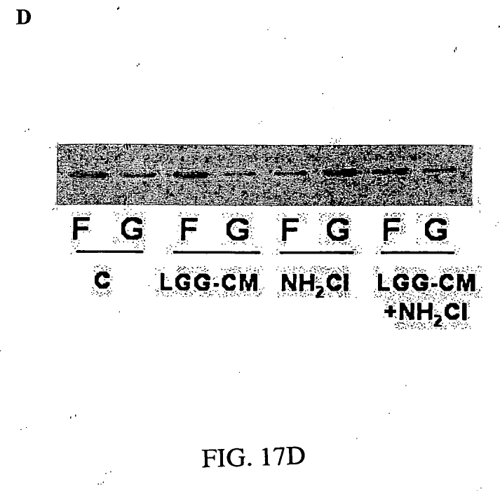

Pretreatment of epithelial cells with LGG-CM provided statistically significant protection against oxidant damage by improving epithelial cell viability in the face of oxidant injury from monochloramine, as demonstrated by chromium release assay (FIGS. 11A and 17A). The protective effects against oxidative damage conferred by LGG-CM were abolished by inhibitors of p38 and JNK, whereas ERK inhibitors had no effect. The data is consistent with a rapid form of Hsp induction through a signaling pathway involving p38 and JNK.

### Example 9

G/F Actin Assays

The capacity of LGG-CM to induce cytoprotective effects in epithelial cells was further explored with an F/G actin assay, another functional readout of the ability of LGG-CM treatment to protect epithelial cells against oxidant stress that more specifically assesses protection against cytoskeletal damage. (FIG. 11B).

Confluent YAMC cell monolayers were switched to 37° C. in IFN-γ-free medium and treated with LGG-CM for one hour, after which the media was replaced or left untreated (control). Cells were left overnight and then treated with the oxidant monochloramine (0.6 mM for 30 minutes) to induce cell injury (Musch et al., 1996; Musch et al., 1999). Cells were rinsed in PBS, harvested, centrifuged (14,000×g for 20 seconds at room temperature) and the pellets were resuspended in 200 μl of 30° C. lysis buffer (1 mM ATP, 50 mM PIPES, pH 6.9, 50 mM NaCl, 5 mM MgCl₂, 5 mM EGTA, 5% (vol/vol) glycerol, 0.1% (vol/vol) Nonidet P-40, Tween 20, and Triton X-100, containing a complete protease inhibitor cocktail). Cells were homogenized by gently pipetting up and down ten times, then incubated at 30° C. for 10 minutes and subjected to centrifugation at 100,000×g for 60 minutes (30° C.). Supernatants were removed for determination of G actin, and pellets (containing F-actin) were resuspended in 200 μl of 4° C. distilled water with 1 μM cytochalasin D and left on ice for 60 minutes. This treatment depolymerized the F-actin fraction so that only the monomeric 45 kDa form would be observed on subsequent Western blots. Afterwards, 20 μl of each extract were removed, Laemmli stop solution was added and the samples were heated to 65° C. for 10 minutes. Samples were resolved on 12.5% polyacrylamide gels by SDS-PAGE and immediately transferred to PVDF membranes (see section on Western blot analysis for additional details). After transfer, immunoblot analysis of actin was performed using a polyclonal anti-actin antibody (Cytoskeleton, Denver, Colo.).

As expected, the untreated controls showed more F than G actin, and pre-treatment with LGG alone did not alter this ratio (FIG. 11B). Treatment of cells with monochloramine (NH₂Cl) caused a shift from the filamentous (F) to the globular (G) form of actin as it disrupted the integrity of the actin cytoskeleton (FIG. 11B). Treatment with LGG-CM prior to monochloramine exposure resulted in preservation of F-actin and partial protection against monochloramine-induced damage to the actin cytoskeleton (FIG. 11B, last two lanes, compare to lanes 5 and 6, reflecting treatment with NH₂CL alone).

To confirm that MAP kinases, specifically p38 and JNK, play a physiologically relevant role in the mechanism of LGG-CM-mediated cytoprotection, cells were treated with inhibitors of the MAP kinases after exposure to LGG-CM and the oxidant injury/chromium release assay was repeated. As expected, less cytoprotection was observed in those cells treated with the MAP kinase inhibitors against p38 and JNK compared to untreated (uninhibited) controls, whereas no differences were observed in cells treated with the ERK inhibitor (FIG. 17B).

To further determine whether Hsp induction was playing an important role in the cytoprotective effects of LGG-CM against oxidant stress and to assess their relative contributions to the cytoprotective effect, siRNA was used to knock down the expression of both Hsp72 and Hsp25 and the chromium release assays were repeated (FIG. 17C, histogram). A Western blot analysis was performed to ensure that decreased Hsp expression had been achieved in each case (FIG. 17C, Western blot). It can be seen that Hsp72 plays a far greater cytoprotective role than does Hsp25 against oxidant stress, as when Hsp72 expression is abolished, most of the protective effect of LGG-CM is lost (FIG. 17C). Hence, the effect of silencing Hsp expression demonstrates that, although Hsp25 may still play a role in the protection afforded by LGG-CM to oxidant injury, Hsp72 plays a greater cytoprotective role than does Hsp25 against oxidant stress.

### Example 10

Akt Assays

Akt is a serine/threonine kinase which plays a pivotal role in cellular proliferation and cell survival. Akt is activated in response to a number of stimuli such as growth factors, and it has been shown to play a role in the regulation of nutrient metabolism (Edinger et al., 2002). LGG-CM also activates Akt in intestinal epithelial cells, and as with MAP kinase activation, this effect is relatively rapid. It is interesting to note that binding of Hsp27 to Akt in COS cells following oxidative stress has been described (Konishi et al., 1997), and one study reports that the Akt-Hsp27 binding interaction is required for Akt activation in neutrophils (Rane et al., 2003).

For Akt assays, YAMC cells were incubated with LGG-CM as described for the MAP kinase assays, except that a concentration of 22.5% LGG-CM was used. Cells were treated for 3 minutes, the LGG-CM-containing medium was removed, fresh medium was added, and cells were either immediately harvested (t=0) or incubated for the indicated times prior to harvest. As a positive control, cells were treated with 100 ng/ml murine TNFα (Peprotech, Rocky Hill, N.J.), a known activator of Akt, for 15 minutes prior to harvest. Akt was inhibited by pretreatment with LY294002 (Cell Signaling, Beverly Mass.), an inhibitor of P13-kinase (which is upstream of Akt and necessary for Akt activation) for 1 hour prior to LGG-CM or TNF treatment. For Western blot analysis of Akt, 15 micrograms of protein per lane were resolved on 10% SDS-PAGE. Samples were transferred onto PVDF membranes, which were then blocked in 5% wt/vol non-fat milk in TBS-Tween for one hour at room temperature. Membranes were incubated overnight at 4° C. with primary antibodies specific for the activated form of Akt with anti-Phospho-Akt (Ser473) (4051S, Cell Signaling), as well as with anti-Akt (9272, Cell Signaling), or anti-Hsc70 (SPA 815, Stressgen). Washes, incubation with horseradish peroxidase-conjugated secondary antibody (Jackson Immunoresearch Labs, Inc., Fort Washington, Pa.), and development of the film using ECL reagent were performed using conventional techniques, as described above.

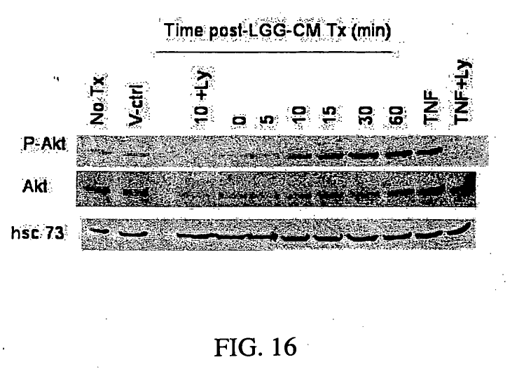

Cells were treated with LGG-CM and examined for activation of Akt, a gene which plays an important role in cell survival. Akt is activated by LGG-CM and this effect is inhibited by treatment with the P13-kinase inhibitor LY (FIG. 16).

### Example 11

Hsp72 ELISA

YAMC cells were grown and treated with LGG DNA as described in Example 1 and then cell lysates were prepared and tested for HSP72 by using an ELISA kit (R&D Systems, Inc., Minneapolis, Minn.) according to the manufacturer's instructions, the only difference being that the individual protease inhibitors for recommended for use in the lysis buffer were substituted with Complete Protease Inhibitor Cocktail (Roche, Mannheim, Germany).

### Example 12

Properties of Bioactive Probiotic Agents

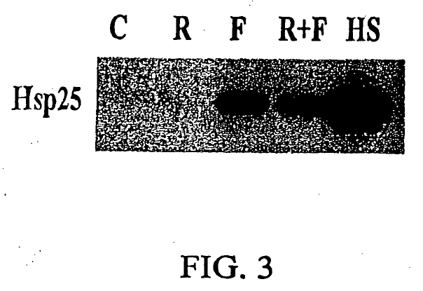

The majority of bioactivity for the LGG-CM appears to reside in compound(s) that are less than 10 kDa. As shown in FIG. 3, the majority of activity, as measured by the ability to induce Hsp25, is present in the filtrate (F) prepared through Centricon filters with a 10 kDa molecular weight cutoff. (See also FIG. 12B.) Contacting cells with the retentate (R) did not induce Hsp25 expression. In addition, recombination of the filtrate and retentate (R+F) did not enhance activity. However, the possibility that larger molecular weight multimer components are required for activity is possible.

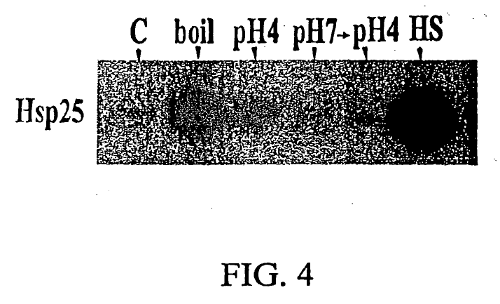

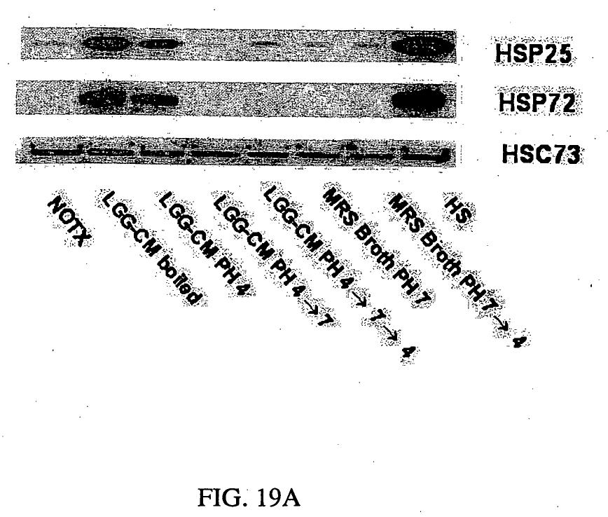

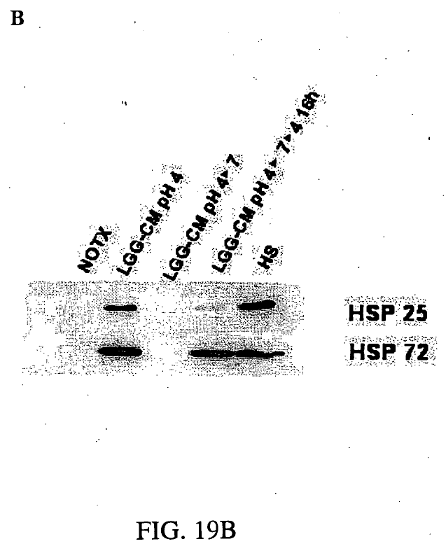

Other properties of the bioactivity of the LGG-CM are its stability in the presence of heat or acid. As shown in FIG. 4 (second lane), LGG-CM retained its bioactivity after boiling for 20 minutes. Additionally, the pH of the LGG-CM was altered and then immediately used to treat the intestinal epithelial cells in order to determine its stability at varying pH (FIG. 19A). Also shown in FIG. 4 (third lane), the LGG-CM was most active at an acidic pH. It should be noted that the pH indicated in FIG. 4 (pH 4) is that of the conditioned media. When added to the bathing media of YAMC cells, a 1:10 dilution occurred and the final pH was between 6.5-6.9, approximating that found in the acid microclimate of the apical membrane of intestinal epithelial cells. When the pH of the conditioned media was adjusted to 7.0, bioactivity was lost (fourth lane). When the pH was readjusted to 4.0 and maintained for 16 hours, activity was not restored (fifth lane), indicating that the active compound(s) was/were unstable at near-neutral pH, although the loss of activity at neutral pH was not absolutely irreversible. At a neutral pH of 7.0, activity of the LGG-CM was abolished (FIG. 19B, lane 4) but if the pH was brought back to pH 4.0 and allowed to equilibrate overnight (FIG. 19B), it was possible to re-establish its Hsp72-inducing ability (compare the absence of Hsp72 induction in the seventh lane in FIG. 19A with the Hsp72 induction in the fourth lane in FIG. 19B, which is almost equivalent in intensity to the heat shock positive control (HS)). If the conditioned media was left to recover for two days at pH 4.0, activity was partially restored, indicating that this effect was not totally irreversible and exposure to near-neutral pH may involve the reversible, partial unfolding or denaturation of the active compound(s).

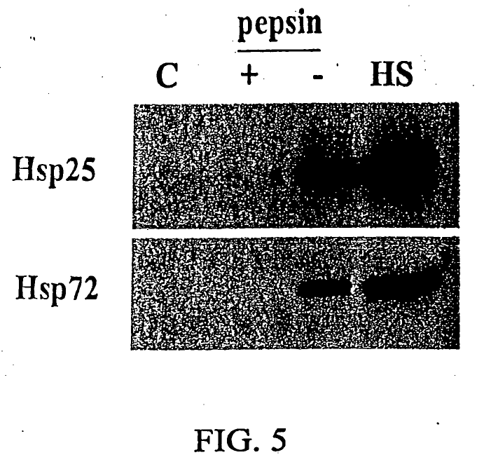

The LGG bioactive compounds were inactivated by the proteolytic enzyme, pepsin. Treatment with pepsin using a standard protocol was followed by filtration of the reaction mixture through a 10 kDa sizing column to remove any residual pepsin. Pepsin was used-in this instance because its activity, in contrast to other proteases, is optimal at acidic pH. As shown in FIG. 5, pepsin treatment of the LGG-conditioned media significantly reduced bioactivity (compare lanes 2 and 3), assessed by induction of Hsp25 and Hsp72. (See also FIG. 12A.) Unfiltered control experiments conducted in parallel established that pepsin itself did not directly affect Hsp expression. (See lanes 5, 6, and 7 of FIG. 12A). These effects were specific, as changes in the constitutive Hsp homolog, Hsc73, were not observed.

The LGG-CM was then subjected to selective ultrafiltration to determine the molecular mass of the active factor. The filtrate (containing molecules of less than 10 kDa) and the retentate (containing molecules larger than 10 kDa) or both together were then used to treat YAMC cells and immunoblots for Hsp25 and Hsp72 were prepared. Only the filtrate (FIG. 18B, lane 3) or both fractions administered together (FIG. 18B, lane 4, R+F) induced Hsp expression in YAMC cells, indicating that the bioactive factor(s) is a protein or peptide of small molecular mass less, i.e., than 10 kDa. Further characterization of the active peptide revealed that it is heat stable, still retaining activity even after boiling (FIG. 18C, compare lanes 2 and 3).

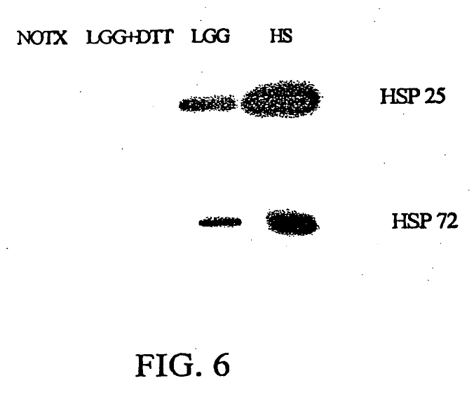

Treatment of LGG-CM with the reducing agent dithiothreitol (DTT) resulted in loss of bioactivity, as illustrated by the loss of inducible Hsp25 and Hsp72 expression by Western blot analysis (FIG. 6). These data indicated that the active compound(s) are protein(s) that are likely to contain cysteine residues and that disulfide bonds may play a critical role in maintaining the secondary structures of the bioactive factor(s).

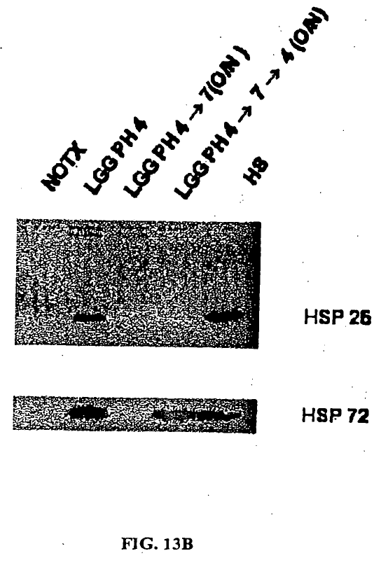

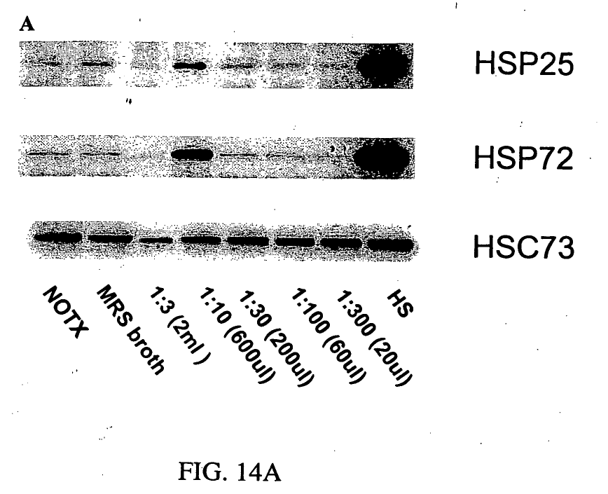

Characterizations of the active peptide revealed that it was also stable at low pH. The pH of the LGG-CM was altered in order to determine its stability at varying pH (FIGS. 13 and 19). At a neutral pH of 7.0, activity of the LGG-CM was abolished if used to treat cells immediately (FIGS. 13A and 19A) but if brought back to pH 4.0 and allowed to equilibrate overnight, it was possible to re-establish its Hsp-inducing ability (FIGS. 13B and 19B). This indicates that the peptide is unstable at pH 7.0 but this instability is not a consequence of irreversible denaturation of the peptide, as returning the LGG-CM to pH 4.0 results in at least partial restoration of the bioactivity (FIGS. 13B and 19B).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein with the same or similar results being achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,534,121
U.S. Pat. No. 6,398,933
Annuk et al., J. Appl. Microbiol., 94:403-412, 2003.
Arvola et al., Pediatrics, 104:e64, 1999.
Besnard et al., Gut, 43(5):634-638, 1998.
Braude and Siemienski, J. Clin. Invest., 47:1763-1773, 1968.
Campierei et al., Gastroenterology, 118:A4179, 2000.
Chang, In: Inflammatory Bowel Diseases, Kirsner (Ed.), Philadelphia, W. B. Saunders Company, 3-19, 1999.
Cleveland et al., Int. J. Food Microbiol., 7.1:1-20, 2001.
Geiss et al., J. Virol., 75(9):4321-4331, 2001.
Gionchetti et al., Gastroenterology, 119:305-309, 2000b.
Gionchetti et al., Gastroenterology, 124:1202-1209, 2003.
Gionchetti et al., J. Gastroenterol. Hepatol., 15:489-493, 2000a.
Goldin et al., Nutr. Cancer, 25: 197-204, 1996.
Goss et al., Proc. Natl. Acad. Sci. USA, 98(9):5116-5121, 2001.
Isolauri et al., Pediatrics, 88:90-97. 1001.
Iyer et al., Science, 283(5398):83-87, 1999.
Kalliomaki et al., Lancet, 362:1869-1871, 2003.
Kojima et al., Gastroenterology, 124:1395-1407, 2003.
Kullisaar et al., Int. J. Food Microbiol., 72:215-224, 2002.
Lee and Goldberg, Mol. Cell. Biol., 18:30-38, 1998.
Lee et al., Appl. Environ. Microbiol., 66:3692-3697, 2000.
Lee et al., J. Med. Microbiol., 52:925-930, 2003.
Leiper et al., Baillieres Clin. Gastroenterol., 12(1):179-99, 1998.
Li and Wong, Proc. Natl. Acad. Sci. USA, 98(1):31-36, 2001.
Liu et al., Am. J. Physiol., 284:C1073-1082, 2003.
Maassen et al., Vaccine, 18:2613-2623, 2000.
Madsen et al., Gastroenterology, 121:580-591, 2001.
Majamaa et al., J. Pediatr. Gastroenterol. Nutr., 20:333-338, 1995.
Makowiec et al., Z. Gastroenterol., 36(8):619-24, 1998.
Mosser et al., Mol. Cell Biol., 8(11):4736-4744, 1988.
Musch et al., Am. J. Physiol., 270:C429-436, 1996.
Musch et al., Gastroenterology, 117:115-122, 1999.
Niedzielin et al., Eur. J. Gastroenterol. Hepatol., 13:1143-1147, 2001.
Ocana et al., Curr. Microbiol., 38:279-284, 1999.
Paraje et al., New Microbiol., 23:423-431, 2000.
Parsell and Lindquist, Annu. Rev. Genet., 27:437-496, 1993.
Podolsky, N. Engl. J. Med., 347:417-429, 2002.
Remington's Pharmaceutical Sciences, 15^{th} ed., pages 1035-1038 and 1570-1580, Mack Publishing Company, Easton, Pa., 1980.
Ropeleski et al., Gastroenterology, 124:1358-1368, 2003.
Schmitz et al., Gastroenterology, 116:301-309, 1999.
Smith, Anal. Biochem., 150:76-85, 1985.
Urayama et al., J. Clin. Invest., 102:1860-1865, 1998.
Whitehead et al., Proc. Natl. Acad. Sci. USA, 90:587-591, 1993.
Wollowski et al., Amer. J. Clinical Nutrition, 73:451S-455S, 2001.
Alak et al., Effect of Lactobacillus reuteri on intestinal resistance to Cryptosporidium parvum infection in a murine model of acquired immunodeficiency syndrome. J Infect Dis 175: 218-221, 1997.
Antony et al., Lactobacillus bacteremia: description of the clinical course in adult patients without endocarditis. Clin Infect Dis 23: 773-778, 1996.
Arvola et al., Prophylactic Lactobacillus GG reduces antibiotic-associated diarrhea in children with respiratory infections: a randomized study. Pediatrics 104: e64, 1999.
Borriello et al., Safety of probiotics that contain lactobacilli or bifidobacteria. Clin Infect Dis 36: 775-780, 2003.
Chang, E., Intestinal epithelial function and response to mucosal injury in inflammatory bowel diseases. In: Inflammatory Bowel Diseases (5th edition ed.), edited by Kirsner J. Philadelphia: W. B. Saunders Company, 1999, p. 3-19.
Cleveland et al., Bacteriocins: safe, natural antimicrobials for food preservation. Int J Food Microbiol 71: 1-20, 2001.
Gabai et al., Hsp70 prevents activation of stress kinases. A novel pathway of cellular thermotolerance. J Biol Chem 272: 18033-18037, 1997.
Goldin et al., The effect of Lactobacillus GG on the initiation and promotion of DMH-induced intestinal tumors in the rat. Nutr Cancer 25: 197-204, 1996.
Grisham et al., Effects of neutrophil-derived oxidants on intestinal permeability, electrolyte transport, and epithelial cell viability. Inflammation 14: 531-542, 1990.
Hamilton-Miller, J M, The role of probiotics in the treatment and prevention of Helicobacter pylori infection. Int J Antimicrob Agents 22: 360-366, 2003.
Hamilton-Miller et al., Deficiencies in microbiological quality and labelling of probiotic supplements. Int J Food Microbiol 72: 175-176, 2002.
Isolauri et al., A human Lactobacillus strain (Lactobacillus casei sp strain GG) promotes recovery from acute diarrhea in children. Pediatrics 88: 90-97, 1991.
Kalliomaki et al., Probiotics and prevention of atopic disease: 4-year follow-up of a randomised placebo-controlled trial. Lancet 361: 1869-1871, 2003.
Keyse, S M, Stress Response: methods and protocols. Totowa: Humana Press, 2000.
Kojima et al., Enteric flora and lymphocyte-derived cytokines determine expression of heat shock proteins in mouse colonic epithelial cells. Gastroenterology 124: 1395-1407, 2003.
Lee et al., Quantitative approach in the study of adhesion of lactic acid bacteria to intestinal cells and their competition with enterobacteria. Appl Environ Microbiol 66: 3692-3697, 2000.
Lee et al., Displacement of bacterial pathogens from mucus and Caco-2 cell surface by lactobacilli. J Med Microbiol 52: 925-930, 2003.
Lister et al., Dipeptide transport and hydrolysis in isolated loops of rat small intestine: effects of stereospecificity. J Physiol 484 (Pt 1): 173-182, 1995.
Liu et al., Protective role of HSP72 against Clostridium difficile toxin A-induced intestinal epithelial cell dysfunction. Am J Physiol Cell Physiol 284: C1073-1082, 2003.
Liu et al., Differential activation of ERK, JNK/SAPK and P38/CSBPIRK map kinase family members during the cellular response to arsenite. Free Radic Biol Med 21: 771-781, 1996.
Madsen et al., Probiotic bacteria enhance murine and human intestinal epithelial barrier function. Gastroenterology 121: 580-591, 2001.
Majamaa et al., Lactic acid bacteria in the treatment of acute rotavirus gastroenteritis. J Pediatr Gastroenterol Nutr 20: 333-338, 1995.
Malin et al., Promotion of IgA immune response in patients with Crohn's disease by oral bacteriotherapy with Lactobacillus GG. Ann Nutr Metab 40: 137-145, 1996.
McNeil et al., Mucosal surface pH of the large intestine of the rat and of normal and inflamed large intestine in man. Gut 28: 707-713, 1987.
Mosser et al., Role of the human heat shock protein Hsp70 in protection against stress-induced apoptosis. Mol Cell Biol 17: 5317-5327, 1997.
Mounier et al., Actin cytoskeleton and small heat shock proteins: how do they interact? Cell Stress Chaperones 7: 167-176, 2002.
Musch et al., Induction of heat shock protein 70 protects intestinal epithelial IEC-18 cells from oxidant and thermal injury. Am J Physiol 270: C429-436, 1996.
Musch et al., Heat-shock protein 72 protects against oxidant-induced injury of barrier function of human colonic epithelial Caco2/bbe cells. Gastroenterology 117: 115-122, 1999.
Parsell et al., The function of heat-shock proteins in stress tolerance: degradation and reactivation of damaged proteins. Annu Rev Genet 27: 437-496, 1993.
Rautio et al., Liver abscess due to a Lactobacillus rhamnosus strain indistinguishable from L. rhamnosus strain GG. Clin Infect Dis 28: 1159-1160, 1999.
Rechkemmer et al., pH-microclimate at the luminal surface of the intestinal mucosa of guinea pig and rat. Pflugers Arch 407: 33-40, 1986.
Reid et al., Potential uses of probiotics in clinical practice. Clin Microbiol Rev 16: 658-672, 2003.
Ropeleski et al., Interleukin-11-induced heat shock protein 25 confers intestinal epithelial-specific cytoprotection from oxidant stress. Gastroenterology 124: 1358-1368, 2003.
Salminen et al., Lactobacillus bacteremia during a rapid increase in probiotic use of Lactobacillus rhamnosus GG in Finland. Clin Infect Dis 35: 1155-1160, 2002.
Salminen et al., Probiotics demonstrating efficacy in clinical settings. Clin Infect Dis 32: 1577-1578, 2001.
Sanderson, I R, The physicochemical environment of the neonatal intestine. Am J Clin Nutr 69: 1028S-1034S, 1999.
Shanahan, F, Probiotics and inflammatory bowel disease: from fads and fantasy to facts and future. Br J Nutr 88 Suppl 1: S5-9, 2002.
Sipsas et al., Safety of Lactobacillus strains used as probiotic agents. Clin Infect Dis 34: 1283-1284; author reply 1284-1285, 2002.
Smith et al., Measurement of protein using bicinchoninic acid. Anal Biochem 150: 76-85, 1985.
Szajewska et al., Efficacy of Lactobacillus GG in prevention of nosocomial diarrhea in infants. J Pediatr 138: 361-365, 2001.
Tuomola et al., Quality assurance criteria for probiotic bacteria. Am J Clin Nutr 73: 393S-398S, 2001.
Urayama et al., Dexamethasone protection of rat intestinal epithelial cells against oxidant injury is mediated by induction of heat shock protein 72. J Clin Invest 102: 1860-1865, 1998.
Vanderhoof et al., Lactobacillus GG in the prevention of antibiotic-associated diarrhea in children. J Pediatr 135: 564-568, 1999.
Wagner et al., Biotherapeutic effects of probiotic bacteria on candidiasis in immunodeficient mice. Infect Immun 65: 4165-4172, 1997.
Whitehead et al., Establishment of conditionally immortalized epithelial cell lines from both colon and small intestine of adult H-2Kb-tsA58 transgenic mice. Proc Natl Acad Sci USA 90: 587-591, 1993.
Yan et al., Probiotic bacterium prevents cytokine-induced apoptosis in intestinal epithelial cells. J Biol Chem 277: 50959-50965, 2002.
Zanke et al., The stress-activated protein kinase pathway mediates cell death following injury induced by cis-platinum, UV irradiation or heat. Curr Biol 6: 606-613, 1996.
Zarate et al., The VP5 domain of VP4 can mediate attachment of rotaviruses to cells. J Virol 74: 593-599, 2000.

What is claimed is:

1. A composition comprising an isolated cytoprotective compound derived from Lactobacillus GG.

2. The composition of claim 1, wherein the cytoprotective compound is derived from a Lactobacillus GG-conditioned medium.

3. The composition of claim 1 wherein the cytoprotective compound protects an epithelial cell from a stress selected from the group consisting of heat and oxidation.

4. The composition of claim 1, wherein the compound induces the expression of at least one heat shock protein.

5. The composition of claim 4, wherein the heat shock protein is selected from the group consisting of Hsp25 and Hsp72.

6. The composition of claim 1, wherein the cytoprotective compound is a protein.

7. The composition of claim 6, wherein the protein is heat stable, acid stable or has a molecular weight of less than 10 kDa.

8. An isolated cytoprotective compound comprising a protein characterized by the following properties:
(a) a capability of being isolated from Lactobacillus GG;
(b) a capability of inducing expression of Hsp25 and Hsp72 in an intestinal epithelial cell;
(c) a molecular weight of less than 10 kDa;
(d) acid stability; and
(e) heat stability.

9. A method for treating a subject with an inflammatory disorder comprising administering to the patient a therapeutically effective dose of an isolated cytoprotective compound derived from a Lactobacillus GG-conditioned medium.

10. The method of claim 9, wherein the subject is a human patient.

11. The method of claim 9, wherein the inflammatory disorder is an inflammatory bowel disease.

12. The method of claim 11, wherein the inflammatory bowel disease is selected from the group consisting of Crohn's disease and ulcerative colitis.

13. The method of claim 10, wherein the compound induces the expression of at least one heat shock protein.

14. The method of claim 13, wherein the heat shock protein is selected from the group consisting of Hsp25 and Hsp72.

15. A method of inducing the expression of at least one of Hsp25 and Hsp72 in a cell comprising contacting the cell with an isolated cytoprotective compound derived from Lactobacillus GG.

16. The method of claim 15, wherein the cytoprotective compound is present in a Lactobacillus GG-conditioned medium.

17. The method of claim 15, wherein the cell is an intestinal epithelial cell.

18. A pharmaceutical composition comprising an isolated cytoprotective compound derived from a Lactobacillus GG-conditioned medium and at least one pharmaceutically acceptable excipient.

19. The pharmaceutical composition of claim 18, wherein the compound induces the expression of at least one heat shock protein.

20. The pharmaceutical composition of claim 18, wherein the heat shock protein is selected from the group consisting of Hsp25 and Hsp72.

21. A method of producing an isolated cytoprotective compound comprising:
(a) obtaining Lactobacillus GG; and
(b) isolating a cytoprotective compound from Lactobacillus GG.

22. The method of claim 21 further comprising culturing the Lactobacillus GG for at least eight hours.

23. The method of claim 21, wherein the cytoprotective compound is a protein.

24. The method of claim 23, wherein the protein is heat stable, acid stable or has a molecular weight of less than 10 kDa.

25. A method for ameliorating a symptom of an inflammatory disorder comprising administering a therapeutically effective dose of the pharmaceutical composition of claim 18 to a subject.

26. The method of claim 25, wherein the subject is a human.

27. The method of claim 25, wherein the inflammatory disorder is an inflammatory bowel disease.

28. The method of claim 27, wherein the inflammatory bowel disease is selected from the group consisting of Crohn's disease and ulcerative colitis.

29. The composition of claim 1, wherein the compound activates a signal transduction pathway in an epithelial cell resulting in expression of a heat shock protein selected from the group consisting of Hsp25 and Hsp72.

30. The composition of claim 29, wherein the activation is mediated by Heat Shock Factor-1 (HSF-1).

31. The composition of claim 29, wherein the signal transduction pathway comprises a kinase selected from the group consisting of MAP kinase, SAP kinase, ERK1 and ERK2.

32. The composition according to claim 31, wherein said pathway activation comprises activation of a kinase selected from MAP kinase, SAP kinase, ERK1 and ERK2.

33. A method of activating a signal transduction pathway in an epithelial cell comprising contacting the cell with an effective amount of the compound according to claim 29.

34. The method of claim 33 wherein the signal transduction pathway comprises a kinase selected from the group consisting of MAP kinase, SAP kinase, ERK1 and ERK2.

35. A method of preventing oxidant injury to a cell comprising administering an effective amount of the compound according to claim 1 to an epithelial cell.

36. A method of stabilizing a cytoskeleton comprising administering an effective amount of the compound according to claim 1 to an epithelial cell.

37. A method of preventing an inflammatory disorder comprising administering a therapeutically effective dose of the pharmaceutical composition of claim 18 to a subject.

38. A kit comprising the pharmaceutical composition of claim 18 and a protocol for administration of the composition to a subject.
